(19) United States
(12) Patent Application Publication
(10) Pub. No.: US 2026/0139227 A1
(43) Pub. Date: May 21, 2026

(54) ADIPOSE-DERIVED REGENERATIVE CELL PREPARATIONS

(71) Applicant: Blue Cell Therapeutics ApS, Copenhagen (DK)

(72) Inventors: Gitte Lund Christensen, Taastrup (DK); Visnja Radulovic, Malmø (SE); Elena Pedraz-Cuesta, Copenhagen (DK); Maja Lind Nybo, Jægerspris (DK); Benjamin Claass, Copenhagen (DK); Jone Marita Kvam, Copenhagen (DK); Søren Paludan Sheikh, Copenhagen (DK); Jakob Lerche Hansen, Copenhagen (DK); Thomas Sandal, Copenhagen (DK); Reza Yarani, Copenhagen (DK)

(21) Appl. No.: 19/441,401

(22) Filed: Jan. 6, 2026

Related U.S. Application Data
(63) Continuation of application No. PCT/IB2025/060979, filed on Oct. 28, 2025.
(60) Provisional application No. 63/712,907, filed on Oct. 28, 2024.
(60) Provisional application No. 63/748,617, filed on Jan. 23, 2025.

(51) Int. Cl. C12N 5/0775

(52) U.S. Cl. CPC C12N 5/0667; C12N 2500/38; C12N 2501/105; C12N 2501/11; C12N 2501/115; C12N 2501/165; C12N 2501/39

(57) ABSTRACT

The present disclosure relates to adipose-derived regenerative cell (ADRC) preparations and methods of culturing ADRCs to obtain ASCs. In particular the present disclosure relates to adipose-derived stromal cells derived from these cultures and their use in treatment.

### CROSS-REFERENCE

This application is a continuation of International Application No. PCT/IB2025/060979, filed Oct. 28, 2025, which claims the benefit of and priority to U.S. Provisional Patent Application No. 63/712,907 filed Oct. 28, 2024, and U.S. Provisional Patent Application No. 63/748,617 filed Jan. 23, 2025, each of which is incorporated by reference in its entirety herein.

### BACKGROUND

Primary adipose-derived regenerative cell (ADRC) preparations are composed of a mixture of cells, including stromal cells, mesenchymal stem cells (MSCs), endothelial cells, vascular smooth muscle cells, and cells of hematopoietic origin.

### SUMMARY

Disclosed herein, in certain embodiments, are cell culture media, comprising: one or more compounds selected from the group consisting of VEGF, EGF, bFGF, IGF, Ascorbic acid, and hydrocortisone or any functional fragments, variants, or homologous proteins of VEGF, EGF, bFGF, or IGF.

In some embodiments, the cell culture medium further comprises one or more bile acids.

In some embodiments, the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA).

In some embodiments, the bile acid is taurocholic acid (TCA).

In some embodiments, the culture medium comprises at least VEGF.

In some embodiments, the culture medium comprises VEGF, EGF, bFGF, IGF,

Ascorbic acid, and hydrocortisone or any functional fragments, variants, or homologous proteins of VEGF, EGF, bFGF, or IGF.

In some embodiments, the culture medium comprises: from about 0.5 ng/ml to about 50 ng/mL VEGF, from about 0.5 ng/ml to about 50 ng/mL EGF, from about 1 ng/ml to about 100 ng/mL bFGF, from about 2 ng/ml to about 200 ng/mL IGF, from about 0.1 μg/mL to about 10 μg/mL Ascorbic acid, or from about 0.02 μg/mL to about 2 μg/mL hydrocortisone.

In some embodiments, the culture medium comprises: from about 0.5 ng/ml to about 50 ng/mL VEGF, from about 0.5 ng/ml to about 50 ng/mL EGF, from about 1 ng/ml to about 100 ng/mL bFGF, from about 2 ng/mL to about 200 ng/ml IGF, from about 0.1 μg/mL to about 10 μg/mL Ascorbic acid, and from about 0.02 μg/mL to about 2 μg/mL hydrocortisone.

In some embodiments, the culture medium comprises: about 0.5 ng/mL VEGF, about 5 ng/mL EGF, about 10 ng/ml bFGF, about 20 ng/ml IGF, about 1 μg/mL Ascorbic acid, or about 0.2 μg/mL hydrocortisone.

In some embodiments, the culture medium comprises: about 0.5 ng/mL VEGF, about 5 ng/mL EGF, about 10 ng/mL bFGF, about 20 ng/ml IGF, about 1 μg/mL Ascorbic acid, and about 0.2 μg/mL hydrocortisone.

In some embodiments, the culture medium is a xenofree medium, optionally wherein the xenofree medium is selected from the group consisting of Miltenyi StemMACS™ MSC Expansion Media and Miltenyi MSC-Brew GMP Medium.

Disclosed herein, in certain embodiments, are mesenchymal stem cells: a) expressing or having increased expression of a protein selected from the group consisting of: ADAM15, ADIRF, AMD1, CDCA8, CDKN2C, CHST11, CXCL5, DKK2, GPALPP1, IGFBP2, LRP11, MEST, MMP1, MT2A, NEO1, NPTX1, PRXL2A, SMOC1, SRGN, ST6GAL2, TNFRSF6B, TPPP3, TRIAP1, ULBP2, VEGFD, CDK1, F5, GINS3, KPNA2, MKI67, NCAPD2, PBK, POLA1, PRG4, SCUBE3, SPDL1, TOP2A, TYMS, UHRF1, and EFHD2 and/or b) having no expression or decreased expression of a protein selected from the group consisting of: AOC3, C2, CFD, CRIP1, F9, IGLV3-19, NTNG1, SERPINB7, SNCA, BGN, CKB, COL12A1, COL14A1, DCN, FBLN1, FCN3, FUCA1, GPLD1, GPNMB, HSPB6, HTRA1, ICAM1, KRT86, LTF, LUM, MX1, POSTN, RAB27B, SERPINB2, SOD2, TGM2, TNC, ALDH2, DPP4, SECTM1, and AKRIC1.

In some embodiments, the mesenchymal stem cell expresses a protein selected from the group consisting of: ADAM15, ADIRF, AMD1, CDCA8, CDKN2C, CHST11, CXCL5, DKK2, GPALPP1, IGFBP2, LRP11, MEST, MMP1, MT2A, NEO1, NPTX1, PRXL2A, SMOC1, SRGN, ST6GAL2, TNFRSF6B, TPPP3, TRIAP1, ULBP2, and VEGFD.

In some embodiments, the mesenchymal stem cell has increased expression of a protein selected from the group consisting of: CDK1, F5, GINS3, KPNA2, MKI67, NCAPD2, PBK, POLA1, PRG4, SCUBE3, SPDL1, TOP2A, TYMS, UHRF1, and EFHD2.

In some embodiments, the mesenchymal stem cell has decreased expression of a protein selected from the group consisting of: BGN, CKB, COL12A1, COL14A1, DCN, FBLN1, FCN3, FUCA1, GPLD1, GPNMB, HSPB6, HTRA1, ICAM1, KRT86, LTF, LUM, MX1, POSTN, RAB27B, SERPINB2, SOD2, TGM2, TNC, ALDH2, DPP4, SECTM1, and AKRIC1.

In some embodiments, the mesenchymal stem cell has no expression of a protein selected from the group consisting of: AOC3, C2, CFD, CRIP1, F9, IGLV3-19, NTNG1, SERPINB7, and SNCA.

In some embodiments, the mesenchymal stem cell: a) expresses or has increased expression of a protein selected from the group consisting of: CXCL5, EFHD2, IGFBP2, MEST, NPTX1, PRG4, SCUBE3, SRGN, ST6GAL2, and TNFRSF6B and/or b) has no expression or decreased expression of a protein selected from the group consisting of: AKRIC1, ALDH2, AOC3, C2, DPP4, GPNMB, IGLV3-19, SECTM1, and SERPINB2.

In some embodiments, the mesenchymal stem cell: a) expresses or has increased expression of SRGN and/or b) has no expression or decreased expression of a protein selected from the group consisting of: AOC3, C2, and IGLV3-19.

In some embodiments, the mesenchymal stem cell: a) expresses or has increased expression of a protein selected from the group consisting of: ADAM15, AMD1, CDCA8, CDK1, CHST11, CXCL5, DKK2, EFHD2, GINS3, KPNA2, MEST, MKI67, MMP1, NCAPD2, NPTX1, PBK, POLA1, SCUBE3, SMOC1, SPDL1, ST6GAL2, TOP2A, TYMS, and UHRF1 and/or b) has no expression or decreased expression of a protein selected from the group consisting of: AKRIC1, ALDH2, CFD, COL14A1, CRIP1, DCN, DPP4, FBLN1, FCN3, FUCA1, GPLD1, GPNMB, HSPB6, KRT86, LUM, MX1, RAB27B, SECTM1, SERPINB2, SOD2, and TGM2.

In some embodiments, the mesenchymal stem cell: a) expresses or has increased expression of a protein selected from the group consisting of: ADAM15, MMP1, and SMOC1 and/or b) has no expression or decreased expression of a protein selected from the group consisting of: CFD, CRIP1, FCN3, and KRT86.

In some embodiments, the mesenchymal stem cell: a) expresses or has increased expression of a protein selected from the group consisting of: AMD1, CDCA8, CDK1, CDKN2C, CHST11, DKK2, EFHD2, F5, GINS3, GPALPP1, IGFBP2, KPNA2, LRP11, MKI67, NCAPD2, NEO1, PBK, POLA1, PRXL2A, SPDL1, ST6GAL2, TNFRSF6B, TOP2A, TPPP3, TYMS, UHRF1, ULBP2, and VEGFD and/or b) has no expression or decreased expression of a protein selected from the group consisting of: AKRIC1, ALDH2, CKB, COL14A1, DPP4, F9, FUCA1, GPLD1, GPNMB, HSPB6, ICAM1, LTF, MX1, NTNG1, RAB27B, SECTM1, SERPINB2, SERPINB7, SNCA, SOD2, and TGM2.

In some embodiments, the mesenchymal stem cell: a) expresses or has increased expression of a protein selected from the group consisting of: CDKN2C, F5, LRP11, NEO1, PRXL2A, TPPP3, ULBP2, and VEGFD and/or b) has no expression or decreased expression of a protein selected from the group consisting of: CKB, F9, ICAM1, LTF, NTNG1, SERPINB7, and SNCA.

In some embodiments, the mesenchymal stem cell: a) expresses or has increased expression of a protein selected from the group consisting of: ADIRF, AMD1, CDCA8, CXCL5, EFHD2, GINS3, GPALPP1, IGFBP2, MT2A, POLA1, PRG4, SCUBE3, SPDL1, ST6GAL2, TNFRSF6B, and TRIAP1 and/or b) has no expression or decreased expression of a protein selected from the group consisting of: AKRIC1, ALDH2, BGN, COL12A1, COL14A1, DCN, DPP4, FBLN1, GPLD1, GPNMB, HTRA1, LUM, POSTN, RAB27B, SECTM1, SERPINB2, and TNC.

In some embodiments, the mesenchymal stem cell: a) expresses or has increased expression of a protein selected from the group consisting of: ADIRF, MT2A, and TRIAP1 and/or b) has no expression or decreased expression of a protein selected from the group consisting of: BGN, COL12A1, HTRA1, POSTN, and TNC.

In some embodiments, the mesenchymal stem cell expresses or has increased expression of a protein selected from the group consisting of: MEST and NPTX1.

In some embodiments, the mesenchymal stem cell expresses or has increased expression of PRG4.

In some embodiments, the mesenchymal stem cell: a) expresses or has increased expression of a protein selected from the group consisting of: DKK2, CHST11, CDK1, KPNA2, MKI67, NCAPD2, PBK, TOP2A, TYMS, and UHRF1 and/or b) has no expression or decreased expression of a protein selected from the group consisting of: FUCA1, HSPB6, MX1, SOD2, and TGM2.

In some embodiments, the mesenchymal stem cell has no expression or decreased expression of a protein selected from the group consisting of: DCN, FBLN1, and LUM.

In some embodiments, the mesenchymal stem cell expresses or has increased expression of GPALPP1.

In some embodiments, the mesenchymal stem cell expresses or has increased expression of a protein selected from the group consisting of: CXCL5 and SCUBE3.

In some embodiments, the mesenchymal stem cell expresses or has increased expression of a protein selected from the group consisting of: IGFBP2 and TNFRSF6B.

In some embodiments, the mesenchymal stem cell: a) expresses or has increased expression of a protein selected from the group consisting of: AMD1, CDCA8, GINS3, POLA1, and SPDL1 and/or b) has no expression or decreased expression of a protein selected from the group consisting of: COL14A1, GPLD1, and RAB27B.

In some embodiments, the mesenchymal stem cell: a) expresses or has increased expression of a protein selected from the group consisting of: ST6GAL2 and EFHD2 and/or b) has no expression or decreased expression of a protein selected from the group consisting of: GPNMB, SERPINB2, ALDH2, DPP4, SECTM1, and AKRIC1.

In some embodiments, the mesenchymal stem cell expresses a cell surface protein or a secreted protein.

In some embodiments, the mesenchymal stem cell: a) expresses or has increased expression of a protein selected from the group consisting of: CXCL5, DKK2, IGFBP2, MMP1, NEO1, NPTX1, SMOC1, SRGN, TNFRSF6B, ULBP2, VEGFD, F5, and SCUBE3 and/or b) has no expression or decreased expression of a protein selected from the group consisting of: AOC3, C2, CFD, F9, IGLV3-19, NTNG1, SNCA, BGN, CKB, COL12A1, COL14A1, DCN, FBLN1, FCN3, GPLD1, HSPB6, HTRA1, LTF, LUM, POSTN, SERPINB2, TGM2, TNC.

In some embodiments, the mesenchymal stem cell: a) expresses or has increased expression of a protein selected from the group consisting of: CXCL5, IGFBP2, NPTX1, SCUBE3, SRGN, and TNFRSF6B and/or b) has no expression or decreased expression of a protein selected from the group consisting of: AOC3, C2, IGLV3-19, and SERPINB2.

In some embodiments, the mesenchymal stem cell: a) expresses or has increased expression of SRGN and/or b) has no expression or decreased expression of a protein selected from the group consisting of: AOC3, C2, and IGLV3-19.

In some embodiments, the mesenchymal stem cell: a) expresses or has increased expression of a protein selected from the group consisting of: CXCL5, DKK2, MMP1, NPTX1, SCUBE3, and SMOC1 and/or b) has no expression or decreased expression of a protein selected from the group consisting of: CFD, COL14A1, DCN, FBLN1, FCN3, GPLD1, HSPB6, LUM, SERPINB2, and TGM2.

In some embodiments, the mesenchymal stem cell: a) expresses or has increased expression of a protein selected from the group consisting of: MMP1 and SMOC1 and/or b) has no expression or decreased expression of a protein selected from the group consisting of: CFD and FCN3.

In some embodiments, the mesenchymal stem cell: a) expresses or has increased expression of a protein selected from the group consisting of: DKK2, F5, IGFBP2, NEO1, TNFRSF6B, ULBP2, and VEGFD and/or b) has no expression or decreased expression of a protein selected from the group consisting of: CKB, COL14A1, F9, GPLD1, HSPB6, LTF, NTNG1, SERPINB2, SNCA, and TGM2.

In some embodiments, the mesenchymal stem cell: a) expresses or has increased expression of a protein selected from the group consisting of: F5, NEO1, ULBP2, and VEGFD and/or b) has no expression or decreased expression of a protein selected from the group consisting of: CKB, F9, LTF, NTNG1, and SNCA.

In some embodiments, the mesenchymal stem cell: a) expresses or has increased expression of a protein selected from the group consisting of: CXCL5, IGFBP2, SCUBE3, and TNFRSF6B and/or b) has no expression or decreased expression of a protein selected from the group consisting of: BGN, COL12A1, COL14A1, DCN, FBLN1, GPLD1, HTRA1, LUM, POSTN, SERPINB2, and TNC.

In some embodiments, the mesenchymal stem cell has no expression or decreased expression of a protein selected from the group consisting of: BGN, COL12A1, HTRA1, POSTN, and TNC.

In some embodiments, the mesenchymal stem cell expresses or has increased expression of NPTX1.

In some embodiments, the mesenchymal stem cell: a) expresses or has increased expression of DKK2 and/or b) has no expression or decreased expression of a protein selected from the group consisting of: HSPB6 and TGM2.

In some embodiments, the mesenchymal stem cell has no expression or decreased expression of a protein selected from the group consisting of: DCN, FBLN1, and LUM.

In some embodiments, the mesenchymal stem cell expresses or has increased expression of a protein selected from the group consisting of: CXCL5 and SCUBE3.

In some embodiments, the mesenchymal stem cell expresses or has increased expression of a protein selected from the group consisting of: IGFBP2 and TNFRSF6B.

In some embodiments, the mesenchymal stem cell has no expression or decreased expression of a protein selected from the group consisting of: COL14A1 and GPLD1.

In some embodiments, the mesenchymal stem cell has no expression or decreased expression of SERPINB2.

In some embodiments, the mesenchymal stem cell is cultured in a medium comprising one or more compounds selected from the group consisting of VEGF, EGF, bFGF, IGF, Ascorbic acid, and hydrocortisone or any functional fragments, variants, or homologous proteins of VEGF, EGF, bFGF, or IGF.

In some embodiments, the medium further comprises one or more bile acids.

In some embodiments, the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA).

In some embodiments, the bile acid is taurocholic acid (TCA).

In some embodiments, the culture medium comprises at least VEGF.

In some embodiments, the culture medium comprises VEGF, EGF, bFGF, IGF, Ascorbic acid, and hydrocortisone or any functional fragments, variants, or homologous proteins of VEGF, EGF, bFGF, or IGF.

In some embodiments, the culture medium comprises: from about 0.5 ng/ml to about 50 ng/mL VEGF, from about 0.5 ng/ml to about 50 ng/mL EGF, from about 1 ng/ml to about 100 ng/mL bFGF, from about 2 ng/ml to about 200 ng/ml IGF, from about 0.1 μg/mL to about 10 μg/mL Ascorbic acid, or from about 0.02 μg/mL to about 2 μg/mL hydrocortisone.

In some embodiments, the culture medium comprises: from about 0.5 ng/ml to about 50 ng/mL VEGF, from about 0.5 ng/mL to about 50 ng/mL EGF, from about 1 ng/ml to about 100 ng/mL bFGF, from about 2 ng/ml to about 200 ng/ml IGF, from about 0.1 μg/mL to about 10 μg/mL Ascorbic acid, and from about 0.02 μg/mL to about 2 μg/mL hydrocortisone.

In some embodiments, the culture medium comprises: about 0.5 ng/mL VEGF, about 5 ng/ml EGF, about 10 ng/ml bFGF, about 20 ng/ml IGF, about 1 μg/mL Ascorbic acid, or about 0.2 μg/mL hydrocortisone.

In some embodiments, the culture medium comprises: about 0.5 ng/mL VEGF, about 5 ng/mL EGF, about 10 ng/mL bFGF, about 20 ng/ml IGF, about 1 μg/mL Ascorbic acid, and about 0.2 μg/mL hydrocortisone.

In some embodiments, the culture medium is a xenofree medium, optionally wherein the xenofree medium is selected from the group consisting of Miltenyi StemMACS™ MSC Expansion Media and Miltenyi MSC-Brew GMP Medium.

Disclosed herein, in certain embodiments, are preparations of ASCs comprising the mesenchymal stem cells described herein.

Disclosed herein, in certain embodiments, are cell cultures comprising (a) an adipose-derived regenerative cell (ADRC) preparation of cells, and (b) a culture medium comprising one or more compounds selected from the group consisting of VEGF, EGF, bFGF, IGF, Ascorbic acid, and hydrocortisone or any functional fragments, variants, or homologous proteins of VEGF, EGF, bFGF, or IGF.

In some embodiments, the culture medium also comprises one or more bile acids.

In some embodiments, the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA).

In some embodiments, the bile acid is taurocholic acid (TCA).

In some embodiments, the culture medium comprises at least VEGF.

In some embodiments, the culture medium comprises VEGF, EGF, bFGF, IGF,

Ascorbic acid, and hydrocortisone or any functional fragments, variants, or homologous proteins of VEGF, EGF, bFGF, or IGF.

In some embodiments, the culture medium comprises: from about 0.5 ng/ml to about 50 ng/mL VEGF, from about 0.5 ng/ml to about 50 ng/mL EGF, from about 1 ng/ml to about 100 ng/mL bFGF, from about 2 ng/ml to about 200 ng/ml IGF, from about 0.1 μg/mL to about 10 μg/mL Ascorbic acid, or from about 0.02 μg/mL to about 2 μg/mL hydrocortisone

In some embodiments, the culture medium comprises: about 0.5 ng/ml VEGF, about 5 ng/mL EGF, about 10 ng/ml bFGF, about 20 ng/ml IGF, about 1 μg/mL Ascorbic acid, or about 0.2 μg/mL hydrocortisone.

In some embodiments, the culture medium is a xenofree medium, optionally wherein the xenofree medium is selected from the group consisting of Miltenyi StemMACS™ MSC Expansion Media and Miltenyi MSC-Brew GMP Medium.

In some embodiments, the ADRC preparation comprises stromal cells, mesenchymal stem cells (MSCs), endothelial cells, vascular smooth muscle cells, and cells of hematopoietic origin.

Disclosed herein, in certain embodiments, are containers comprising the cell cultures described herein.

Disclosed herein, in certain embodiments, are methods of manufacturing an ASC preparation, comprising: a) providing an ADRC preparation and a culture medium comprising one or more compounds selected from the group consisting of VEGF, EGF, bFGF, IGF, Ascorbic acid, and hydrocortisone or any functional fragments, variants, or homologous proteins of VEGF, EGF, bFGF, or IGF; and b) contacting the ADRC preparation with the culture medium for a time sufficient to allow growth of the MSCs in the ADRC preparation, thereby providing adipose-derived stromal cells (ASCs).

In some embodiments, the methods further comprise enriching the MSCs/ASCs from the ADRC preparation.

In some embodiments, the methods further comprise enriching factors or extracellular vesicles released from the ASCs, thereby providing a preparation of factors or extracellular vesicles released from the ASCs.

In some embodiments, the culture medium further comprises one or more bile acids.

In some embodiments, the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA).

In some embodiments, the bile acid is taurocholic acid (TCA).

In some embodiments, the culture medium comprises at least VEGF.

In some embodiments, the culture medium comprises VEGF, EGF, bFGF, IGF, Ascorbic acid, and hydrocortisone or any functional fragments, variants, or homologous proteins of VEGF, EGF, bFGF, or IGF.

In some embodiments, the culture medium comprises: from about 0.5 ng/ml to about 50 ng/mL VEGF, from about 0.5 ng/ml to about 50 ng/mL EGF, from about 1 ng/ml to about 100 ng/mL bFGF, from about 2 ng/ml to about 200 ng/ml IGF, from about 0.1 μg/mL to about 10 μg/mL Ascorbic acid, or from about 0.02 μg/mL to about 2 μg/mL hydrocortisone.

In some embodiments, the culture medium comprises: from about 0.5 ng/ml to about 50 ng/mL VEGF, from about 0.5 ng/ml to about 50 ng/mL EGF, from about 1 ng/ml to about 100 ng/mL bFGF, from about 2 ng/mL to about 200 ng/ml IGF, from about 0.1 μg/mL to about 10 μg/mL Ascorbic acid, and from about 0.02 μg/mL to about 2 μg/mL hydrocortisone.

In some embodiments, the culture medium comprises: about 0.5 ng/mL VEGF, about 5 ng/ml EGF, about 10 ng/mL bFGF, about 20 ng/mL IGF, about 1 μg/mL Ascorbic acid, or about 0.2 μg/mL hydrocortisone.

In some embodiments, the culture medium comprises: about 0.5 ng/mL VEGF, about 5 ng/mL EGF, about 10 ng/ml bFGF, about 20 ng/ml IGF, about 1 μg/mL Ascorbic acid, and about 0.2 μg/mL hydrocortisone.

In some embodiments, the culture medium is a xenofree medium, optionally wherein the xenofree medium is selected from the group consisting of Miltenyi StemMACS™ MSC Expansion Media and Miltenyi MSC-Brew GMP Medium.

In some embodiments, the ADRC preparation is isolated from a lipoaspirate obtained from a liposuction.

In some embodiments, the ADRC preparation is mechanically isolated or chemically isolated.

In some embodiments, the ADRC preparation is treated with collagenase.

In some embodiments, the ADRC preparation comprises stromal cells, mesenchymal stem cells (MSCs), endothelial cells, vascular smooth muscle cells, and cells of hematopoietic origin.

In some embodiments, the culture medium is exchanged for every 3-4 days.

In some embodiments, the compounds and/or bile acids are replenished by addition directly to the media.

In some embodiments, the method is performed in a 2-D culture.

In some embodiments, the method is performed using microcarriers as a growth surface.

In some embodiments, the method is performed in a 3-D culture.

In some embodiments, the ADRC preparation is cultured for at least 2 passages, at least 5 passages, or at least 10 passages.

In some embodiments, the ADRC preparation is cultured for at least 5 passages.

In some embodiments, the ADRC preparation is cultured for 5 passages.

In some embodiments, the ADRC preparation is cultured for at least 6, at least 8, at least 10, at least 15, or at least 20 cumulative population doublings (cPDs).

In some embodiments, the ADRC preparation is cultured for at least 6 cPDs.

In some embodiments, the ADRC preparation is cultured for at least 12 cPDs.

In some embodiments, the time to reach 70-80% confluency does not exceed 160 hours, does not exceed 150 hours, or does not exceed 140 hours.

Disclosed herein, in certain embodiments, are preparations of ASCs obtained by the methods described herein.

Disclosed herein, in certain embodiments, are containers comprising the ASC preparations described herein.

Disclosed herein, in certain embodiments, are preparations of ASCs for use as a medicament.

Disclosed herein, in certain embodiments, are preparations of ASCs for use in the treatment of erectile dysfunction.

In some embodiments, the treatment is an allogenic treatment.

Disclosed herein, in certain embodiments, are preparations of regenerative factors or extracellular vesicles released from ASCs obtained by the methods described herein.

Disclosed herein, in certain embodiments, are mesenchymal stem cells obtained by the methods described herein.

Disclosed herein, in certain embodiments, are mesenchymal stem cells expressing increased expression and/or activity of angiogenic factors, neurogenic factors, or both angiogenic factors and neurogenic factors compared to uncultured cells or cells cultured in a culture medium that does not comprise VEGF, EGF, bFGF, IGF, Ascorbic acid, and hydrocortisone or any functional fragments, variants, or homologous proteins of VEGF, EGF, bFGF, or IGF.

In some embodiments, the culture medium does not comprise one or more bile acids.

In some embodiments, the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA).

In some embodiments, the bile acid is taurocholic acid (TCA).

Disclosed herein, in certain embodiments, are ASC populations comprising more than about 95% mesenchymal stem cells expressing increased expression and/or activity of angiogenic factors, neurogenic factors, or both angiogenic factors and neurogenic factors.

In some embodiments, the ASC population does not comprise stromal cells, endothelial cells, smooth muscle cells, or cells of hematopoietic origin.

In some embodiments, cells of the ASC population do not express adipose markers.

In some embodiments, the mesenchymal stem cells express increased expression and/or activity of angiogenic factors, neurogenic factors, or both angiogenic factors and neurogenic factors as compared to uncultured cells or cells cultured in a culture medium that does not comprise VEGF, EGF, bFGF, IGF, Ascorbic acid, and hydrocortisone or any functional fragments, variants, or homologous proteins of VEGF, EGF, bFGF, or IGF.

In some embodiments, the culture medium does not comprise one or more bile acids.

In some embodiments, the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA).

In some embodiments, the bile acid is taurocholic acid (TCA).

### DETAILED DESCRIPTION

Mesenchymal stem cells secrete a wide spectrum of angiogenic, neurogenic, and anti-inflammatory growth factors (collectively, “regenerative factors”) at the site of an injury. The regenerative factors released from the MSCs promote vascular and neuronal regeneration and protection, for example, by stimulating endothelial cell growth and function or enhancing neuronal survival, respectively or in combination. These regenerative factors may be released in extracellular vesicles, which can be isolated from the culture medium.

Disclosed herein, in certain embodiments, are methods of treating diseases or disorders characterized by insufficient or abnormal vessel growth or postoperative damage to vessels and/or neurons resulting in reduced blood flow and hypoxia and/or nerve damage, comprising administering a composition comprising human adipose-derived stromal cells (ASCs). In some embodiments, the disease or disorder is erectile dysfunction.

Disclosed herein, in certain embodiments, are standard culture conditions useful in generating human ASCs from a population of human ADRCs.

Disclosed herein, in certain embodiments, are cell cultures comprising an expanded population of mesenchymal stem cells (MSCs), derived from a human adipose, wherein the MSCs adhere to culture-compatible surfaces under standard culture conditions.

Disclosed herein, in certain embodiments, culture-compatible surfaces are such as and not limited to, e.g. synthetic or plastic substrates, polymeric materials, or extracellular matrix-mimicking coatings.

Disclosed herein, in certain embodiments, are cell cultures of mesenchymal stem cells (MSCs), derived from human adipose tissue and expanded in vitro.

Disclosed herein, in certain embodiments, are methods of generating human ASC populations, comprising contacting a population of ADRCs adhered to culture-compatible surfaces under standard culture conditions useful in generating human ASCs.

Disclosed herein, in certain embodiments, are cell culture mediums useful in generating human ASCs from a population of human ADRCs.

Disclosed herein, in certain embodiments, are cell cultures comprising: (a) a human adipose-derived regenerative cell (ADRC) population, and (b) cell culture mediums useful in generating human ASCs.

Disclosed herein, in certain embodiments, are methods of generating human ASC populations, comprising contacting a population of ADRCs with a cell culture medium useful in generating human ASCs.

### Definitions

Primary adipose-derived regenerative cell (ADRC) preparations refer to cell populations (e.g., non-adipocytes) derived from adipose tissue and composed of a mixture of cells, including stromal cells, mesenchymal stem cells (MSCs), endothelial cells, vascular smooth muscle cells, and cells of hematopoietic origin. ADRCs may be obtained from a lipoaspirate such as obtained from a liposuction.

Stromal vascular fraction (SVF) refers to the non-adipocyte cell fraction from adipose tissue, e.g., ADRCs.

Enriching refers to the process of obtaining mesenchymal stem cells using the culture conditions described herein, e.g., by selective adherence, adaptation to/modification to culture conditions and culture expansion.

Cumulative population doublings (cPD) are the average number of doublings a cell population undergoes during cell culture. It is a measure of cellular expansion, and for 2D culture, it is calculated by the sum of the population doublings that occur for each passage.

Mesenchymal stem cell (MSC) refers to cells meeting the following criteria:
1) MSCs must be plastic-adherent when maintained in standard culture conditions;
2) MSCs must express (a) high levels (such as above 95% positive in a population of cells) of CD105, CD73, and CD90 and (b) low levels (such as below 5% positive in a population of cells) of the markers CD45 and/or HLA-DR.

Adipose-derived stromal cells (ASC) preparation refers to mesenchymal stem cell (MSC) enriched cell population compared to the initial adipose-derived regenerative cell (ADRC) isolation.

Regenerative factors refers to angiogenic and neurogenic factors released by MSCs. In some embodiments, the regenerative factors are released in extracellular vesicles. When regenerative factors are mentioned here it is understood as free soluble regenerative factors or regenerative factors inside extracellular vesicles.

Neurogenic is used interchangeable with neurotrophic and neuroregenerative. For example, neurogenic factors refers to neurotrophic factors.

Endothelial cell markers refers to at least CD31, CD49f, and ESAM.

Hematopoietic cell marker refers to at least CD45.

Immunogenic cell marker refers to at least HLA-DR.

Growth factor cocktail refers to compounds plus TCA

IGF is synonymous with IGF-1.

Absent refers to whether a gene or protein is not detectable in a cell population as compared to a different cell population. For example, absent refers to whether a gene or protein is not detectable and a numerical value cannot be assigned in the cell population cultured using a condition as compared to a cell population grown using a different condition.

Present refers to whether a gene or protein is detectable in a cell population as compared to a different cell population. For example, present refers to whether a gene or protein is detectable and a numerical value can be assigned in the cell population cultured using a condition as compared to a cell population grown using a different condition.

Downregulated refers to a gene or protein being expressed at least [LOG₂]₃-fold lower in a cell population as compared to a different cell population. For example, downregulated refers to whether a gene or protein is expressed lower in the cell population cultured using a condition as compared to a cell population grown using a different condition.

Upregulated refers to a gene or protein being expressed at least [LOG₂]₃-fold higher in a cell population as compared to a different cell population. For example, upregulated refers to whether a gene or protein is expressed higher in the cell population cultured using a condition as compared to a cell population grown using a different condition.

### BRIEF DESCRIPTION OF THE FIGURES

FIGS. 1A-1B show adipose-derived regenerative cells (ADRCs) grow faster in MV2 cell media (FIG. 1B) compared to DMEM (FIG. 1A) with 10% FBS, which is a standard media for mesenchymal stem cells. Accumulated cell numbers are calculated as fold change starting from 1 cell.

FIG. 2A shows adipose-derived regenerative cell (ADRC) growth in xenofree MSC media (XF), MV2 media from Promocell without (MV2+2% FBS) or with compounds added (MV2+2% FBS+compounds). Growth was evaluated by automated imaging based confluence determination.

FIG. 2B shows cells in xenofree media and cells in MV2+2% FBS+compounds were cultured for 4-7 passages. Accumulated cell numbers are shown.

FIG. 2C shows cells from 3 independent donors cultured in xenofree media for 9 passages. Accumulated cell numbers are shown.

FIG. 2D shows doubling time for cell growth of the same 3 donors as depicted in FIG. 2C (p=passage).

FIGS. 3A-3B show adipose-derived regenerative cell growth in xenofree MSC media (XF), xenofree media with compounds (XF+compounds), and xenofree media with compounds and TCA (XF+compounds+TCA). Growth of cells from two donors is depicted. X ####indicates fold change obtained per seeded cell, and is equal to the Y-axis.

FIGS. 4A-4B show evaluation of mesenchymal stem cell (FIG. 4A) and endothelial surface and immunogenicity marker HLA-DR (FIG. 4B) marker positivity in passaged ADRCs.

FIGS. 5A-5E show angiogenic properties of adipose-derived regenerative cells (ADRCs) cultured in 3 passages (#) in xenofree MSC media (XF), xenofree media with compounds (XF+compounds) or xenofree media with compounds and 10 μm taurocholic acid (XF+compounds+TCA). FIG. 5A shows percent wound healing (y-axis). Vertical scratches are made in the cell layer using BioTek AutoScratch. The conditioned media from the ADRCs was transferred to the scratched HMEC-1 cells plate. Image analysis was used to calculate the area of the scratch at timepoint 0 hours and after 18 hours. Wound healing was calculated as percentage wound closure/healing. FIG. 5B shows data from an aortic ring assay. Aortic rings from 4-6 mice were cut in 1 mm pieces and placed in a matrigel matrix and co-cultured with ADRCs seeded on inserts for 6 days. Images of induced sprouting were taken after 6 days. Images were inverted and black pixels in the selected area were quantified and normalized to control mean area for each individual mouse. FIG. 5C shows an image of aortic ring and inverted picture of sprouts used for quantification. FIG. 5D shows a graph of angiogenic properties, indicated as CD31-positive mouse cells/mg Geltrex plug, of ADRCs cultured in 3 passages (p) in xenofree MSC media (XF), xenofree media with compounds (XF+compounds) or xenofree media with compounds and 10 μm taurocholic acid (XF+compounds+TCA). FIG. 5E shows data from an aortic ring assay. Aortic rings from 3-6 mice were cut in 0.5 mm rings and placed in a matrigel matrix for co-culturing with ADRCs seeded on the same chamber. Images of induced sprouting were taken after 5 days. Images were inverted and black pixels in the selected area were quantified and normalized to control mean area for each individual mouse. Different points (with different shapes indicates data from independent growth experiments).

FIGS. 6A-6B show graphs of total neurite length in cultures of neurons isolated from dorsal root ganglia stimulated with no FBS (FIG. 6A) or 0.5% FBS (FIG. 6B). Neurons were cultured in the presence of conditioned media harvested from growth factor-free ASC cultures or in growth factor-free control media that was never exposed to any ASC culture.

FIG. 7 shows a bar plot of enriched Gene Ontology biological process terms ranked by significance for the input gene set, showing the top pathways by-log 10 (adjusted P value) on the y-axis and GO term descriptions on the x-axis. Taller bars indicate more significant enrichment after multiple-testing correction using Benjamini-Hochberg procedures commonly applied in enrichment workflows.

FIGS. 8A-8D show gene expression data of cells treated with XF media and XF growth factor cocktail (i.e., XF+compounds+TCA media) for genes EREG, HGF, NRCAM, and TMEFF2 (FIG. 8A); CXCL12, SEMA3A, CALCRL, and FGFR2 (FIG. 8B); IGFBP2, CXCL8 (IL8), ICAM1, and PECAM1 (FIG. 8C); and APOD and ICAM5 (FIG. 8D).

FIG. 9 shows EREG and HGF expression after certain number of passages in culture with XF+compounds+TCA media.

FIG. 10 shows VEGFA, FGF2, and DKK3 expression after certain number of passages in culture with XF+compounds+TCA media.

FIG. 11 shows BDNF and GDNF expression after certain number of passages in culture with XF+compounds+TCA media.

FIG. 12 shows DKK3 (pg/mL) in starving culture supernatant after culture with control media (media ctrl) or after certain number of passages in culture with XF+compounds+TCA media.

FIG. 13 shows NRCAM, ICAM1, and TMEFF2 expression after certain number of passages in culture with XF+compounds+TCA media.

FIG. 14 shows angiogenic properties, indicated as CD31-high mouse cells/mg Geltrex plug, of adipose-derived stem cell cultured for 4 passages (p4) in xenofree MSC media (XF) or of non-cultured cells from stromal vascular fraction (SVF #0) isolate.

FIG. 15 shows angiogenic properties, indicated as fold increase of area covered by sprouts, of adipose-derived stem cells cultured with the indicated number of cumulative population doublings (cPDs). A linear regression trend line was calculated for cells cultured in xenofree MSC media (XF) or xenofree media with compounds and TCA for 4 different donors.

FIG. 16 shows angiogenic properties, indicated as CD31-positive mouse cells/mg Geltrex plug, of adipose-derived stem cell cultured for 3 passages (p3) in xenofree MSC media (XF) or xenofree media with compounds and TCA (XF+compounds+TCA) for two different donors.

FIG. 17 shows angiogenic properties, indicated as fold increase of area covered by sprouts from Corpus Cavernosum explants, of ASCs from culture in XF with compounds and TCA or from ADRCS, i.e. non-cultured cells from stromal vascular fraction (SVF) isolate.

FIG. 18A summarizes data from Step 5 of Example 9 in which cluster with >5,000 cells was selected and a pseudo bulk RNAseq count matrix was created.

FIG. 18B summarizes data from Step 6 of Example 9 in which sequencing depth distribution of SC dataset was calculated and a pseudo-SC dataset from pseudo bulk RNA dataset was generated.

FIG. 18C summarizes data from Step 7 of Example 9 in which pseudo-SC dataset from pseudo bulk dataset was mapped to full SC dataset.

FIG. 19 shows data demonstrating BCT cell types as compared to donor cells.

FIGS. 20A-20E show representative trace plots of the nerve injury-induced erectile dysfunction animal model with measured intracavernous pressure (ICP, bottom trace) and arterial pressure (top trace) for a healthy animal (FIG. 20A), and four animals (FIGS. 20B-20E) that has undergone bilateral cavernous nerve injury (BCNI) and treated with either vehicle (FIG. 20B) or three different doses of ASC at passage 5 (p5) (FIGS. 20C-20E). Stimulation of the cavernous nerve (CN) is performed for 1 minute from 20 seconds on the y-axis at 1.5 mA, 16 Hz, 6 V, and pulse width at 5 milliseconds.

FIG. 21 shows the effect of ASC and stromal vascular fraction (SVF) treatment of erectile function (ICP) following an nerve injury-induced erectile dysfunction rat model (bilateral cavernous nerve injury (BCNI)). The area under the curve of the intracavernous pressure normalized to mean arterial pressure (AUC-ICP/MAP) is depicted for each treatment group: Healthy, BCNI+vehicle, BCNI+ASC (0.25, 1.00, and 2.50×10E6 cells), and BCNI+SVF (1.00 and 2.50×10E6 cells). Data are presented as mean±standard deviation. Statistical significance was determined by one-way ANOVA, with post hoc comparisons indicating significant improvements, as indicated by asterisks.

FIG. 22A shows a representative picture of a CD31-stained section of the corpus cavernosum, with the urethra intensely colored at the top (arrow) and the penile vein at the bottom (arrow head). The corpus cavernosum (region of interest) is marked in black.

FIG. 22B represents the semi-automated quantification of the CD31-positive area out of the total corpus cavernosum in a transverse section of the penis. Data are presented as mean±standard deviation.

### CELL CULTURE MEDIUM

Disclosed herein, in certain embodiments are cell culture mediums useful in generating human ASCs from a population of human ADRCs. In some embodiments, the culture medium comprises one or more compounds selected from the group consisting of VEGF, EGF, bFGF, IGF, Ascorbic acid, and hydrocortisone or any functional fragments, variants, or homologous proteins of VEGF, EGF, bFGF, or IGF.

In some embodiments, the cell culture medium comprises VEGF. In some embodiments, the VEGF is able to increase the growth rate of the MSCs from a population of ADRCs.

In some embodiments, the culture medium comprises VEGF, EGF, bFGF, IGF, Ascorbic acid, and hydrocortisone or any functional fragments, variants, or homologous proteins of VEGF, EGF, bFGF, or IGF.

In some embodiments, the culture medium also comprises one or more bile acids. In some embodiments, the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA). In some embodiments of the present disclosure, the bile acid is taurocholic acid (TCA).

In some embodiments, a suitable variant of Ascorbic acid is 2-phospho-ascorbic acid.

In some embodiments, a suitable variant of VEGF is Vascular Endothelial Growth Factor 165.

In some embodiments, a suitable variant of IGF is Long R3 IGF.

The concentrations of the components can be applied over a broad range. In some embodiments, the culture medium comprises a range of VEGF, EGF, bFGF, IGF, Ascorbic acid, and hydrocortisone or any functional fragments, variants, or homologous proteins of VEGF, EGF, bFGF, or IGF.

In some embodiments, the culture medium comprises from about 0.1 ng/ml to about 50 ng/mL VEGF or any functional fragments, variants, or homologous proteins of VEGF. In some embodiments, the culture medium comprises from about 0.5 ng/mL to about 50 ng/mL VEGF or any functional fragments, variants, or homologous proteins of VEGF. In some embodiments, the culture medium comprises about 0.1 ng/ml, about 0.25 ng/ml, about 0.5 ng/ml, about 1 ng/mL, about 2 ng/mL, about 3 ng/mL, about 4 ng/ml, about 5 ng/ml, about 6 ng/ml, about 7 ng/ml, about 8 ng/ml, about 9 ng/ml, about 10 ng/ml, about 12 ng/ml, about 14 ng/ml, about 16 ng/mL, about 18 ng/ml, about 20 ng/ml, about 25 ng/ml, about 30 ng/ml, about 35 ng/ml, about 40 ng/ml, about 45 ng/ml, or about 50 ng/ml VEGF or any functional fragments, variants, or homologous proteins of VEGF. In some embodiments, the culture medium comprises from about 0.1 ng/ml to about 1 ng/ml, from about 0.1 ng/ml to about 0.5 ng/mL, from about 0.5 ng/ml to about 5 ng/ml, from about 5 ng/ml to about 10 ng/mL, from about 10 ng/ml to about 15 ng/ml, from about 15 ng/ml to about 20 ng/mL, from about 20 ng/mL to about 25 ng/mL, from about 25 ng/ml to about 30 ng/mL, from about 30 ng/mL to about 35 ng/mL, from about 35 ng/ml to about 40 ng/ml, from about 40 ng/ml to about 45 ng/ml, or from about 45 ng/ml to about 50 ng/ml. In some embodiments, the culture medium comprises about 0.5 ng/mL VEGF or any functional fragments, variants, or homologous proteins of VEGF.

In some embodiments, the culture medium comprises from about 0.5 ng/ml to about 50 ng/mL EGF or any functional fragments, variants, or homologous proteins of EGF. In some embodiments, the culture medium comprises about 0.5 ng/ml, about 1 ng/mL, about 2 ng/ml, about 3 ng/ml, about 4 ng/mL, about 5 ng/ml, about 6 ng/ml, about 7 ng/ml, about 8 ng/ml, about 9 ng/ml, about 10 ng/ml, about 12 ng/ml, about 14 ng/ml, about 16 ng/ml, about 18 ng/mL, about 20 ng/ml, about 25 ng/ml, about 30 ng/mL, about 35 ng/ml, about 40 ng/ml, about 45 ng/mL, or about 50 ng/mL EGF or any functional fragments, variants, or homologous proteins of EGF. In some embodiments, the culture medium comprises from about 0.1 ng/ml to about 1 ng/mL, from about 0.1 ng/ml to about 0.5 ng/ml, from about 0.5 ng/mL to about 5 ng/mL, from about 5 ng/ml to about 10 ng/ml, from about 10 ng/ml to about 15 ng/mL, from about 15 ng/ml to about 20 ng/ml, from about 20 ng/mL to about 25 ng/ml, from about 25 ng/mL to about 30 ng/ml, from about 30 ng/ml to about 35 ng/ml, from about 35 ng/ml to about 40 ng/ml, from about 40 ng/mL to about 45 ng/ml, or from about 45 ng/ml to about 50 ng/mL EGF or any functional fragments, variants, or homologous proteins of EGF. In some embodiments, the culture medium comprises about 5 ng/mL EGF or any functional fragments, variants, or homologous proteins of EGF.

In some embodiments, the culture medium comprises from about 1 ng/ml to about 100 ng/mL bFGF or any functional fragments, variants, or homologous proteins of bFGF. In some embodiments, the culture medium comprises about 1 ng/ml, about 5 ng/ml, about 10 ng/mL, about 12 ng/ml, about 14 ng/mL, about 16 ng/mL, about 18 ng/ml, about 20 ng/mL, about 25 ng/ml, about 30 ng/mL, about 35 ng/ml, about 40 ng/mL, about 45 ng/ml, about 50 ng/ml, about 55 ng/mL, about 60 ng/ml, about 65 ng/mL, about 70 ng/mL, about 75 ng/mL, about 80 ng/ml, about 85 ng/mL, about 90 ng/mL, about 95 ng/ml, or about 100 ng/mL bFGF or any functional fragments, variants, or homologous proteins of bFGF. In some embodiments, the culture medium comprises from about 1 ng/ml to about 100 ng/ml, from about 5 ng/ml to about 100 ng/ml, from about 10 ng/ml to about 100 ng/ml, from about 10 ng/ml to about 90 ng/mL, from about 10 ng/ml to about 80 ng/ml, from about 10 ng/ml to about 70 ng/mL, from about 10 ng/ml to about 60 ng/mL, from about 10 ng/ml to about 50 ng/ml, from about 10 ng/mL to about 40 ng/mL, from about 10 ng/ml to about 30 ng/ml, or from about 10 ng/ml to about 20 ng/mL bFGF or any functional fragments, variants, or homologous proteins of bFGF. In some embodiments, the culture medium comprises about 10 ng/mL bFGF or any functional fragments, variants, or homologous proteins of bFGF.

In some embodiments, the culture medium comprises from about 2 ng/ml to about 200 ng/mL IGF or any functional fragments, variants, or homologous proteins of IGF. In some embodiments, the culture medium comprises about 1 ng/ml, about 2 ng/ml, about 5 ng/ml, about 10 ng/ml, about 20 ng/ml, about 25 ng/ml, about 30 ng/ml, about 35 ng/ml, about 40 ng/mL, about 45 ng/mL, about 50 ng/mL, about 55 ng/ml, about 60 ng/ml, about 65 ng/ml, about 70 ng/mL, about 75 ng/ml, about 80 ng/ml, about 85 ng/ml, about 90 ng/ml, about 95 ng/mL, about 100 ng/ml, about 110 ng/ml, about 120 ng/ml, about 130 ng/mL, about 140 ng/mL, about 150 ng/ml, about 160 ng/mL, about 170 ng/ml, about 180 ng/ml, about 190 ng/mL, or about 200 ng/mL IGF or any functional fragments, variants, or homologous proteins of IGF. In some embodiments, the culture medium comprises from about 5 ng/ml to about 200 ng/ml, from about 20 ng/ml to about 200 ng/ml, from about 50 ng/ml to about 200 ng/mL, from about 100 ng/ml to about 200 ng/mL, from about 10 ng/ml to about 150 ng/mL, from about 50 ng/ml to about 150 ng/mL, from about 100 ng/ml to about 150 ng/mL, from about 10 ng/mL to about 100 ng/mL, from about 10 ng/ml to about 80 ng/mL, from about 10 ng/ml to about 50 ng/ml, from about 10 ng/mL to about 40 ng/mL, or from about 10 ng/ml to about 20 ng/ml IGF or any functional fragments, variants, or homologous proteins of IGF. In some embodiments, the culture medium comprises about 20 ng/ml IGF or any functional fragments, variants, or homologous proteins of IGF.

In some embodiments, the culture medium comprises from about 0.1 μg/mL to about 10 μg/mL Ascorbic acid. In some embodiments, the culture medium comprises about 0.1 μg/mL, about 0.25 μg/mL, about 0.5 μg/mL, about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, or about 10 μg/mL Ascorbic acid. In some embodiments, the culture medium comprises from about 0.1 μg/mL to about 10 μg/mL, from about 0.1 μg/mL to about 8 μg/mL, from about 0.1 μg/mL to about 5 μg/mL, from about 0.1 μg/mL to about 1 μg/mL, from about 0.1 μg/mL to about 0.5 μg/mL, from about 0.5 μg/mL to about 10 μg/mL, from about 0.5 μg/mL to about 8 μg/mL, from about 0.5 μg/mL to about 5 μg/mL, from about 0.5 μg/mL to about 4 μg/mL, from about 0.5 μg/mL to about 2 μg/mL, from about 1 μg/mL to about 10 μg/mL, from about 1 μg/mL to about 8 μg/mL, from about 1 μg/mL to about 5 μg/mL, from about 2 μg/mL to about 10 μg/mL, from about 2 μg/mL to about 8 μg/mL, from about 2 μg/mL to about 5 μg/mL, or from about 5 μg/mL to about 10 μg/mL Ascorbic acid. In some embodiments, the culture medium comprises about 1 μg/mL Ascorbic acid.

In some embodiments, the culture medium comprises from about 0.02 μg/mL to about 2 μg/mL hydrocortisone. In some embodiments, the culture medium comprises about 0.01 μg/mL, about 0.02 μg/mL, about 0.05 μg/mL, about 0.1 μg/mL, about 0.2 μg/mL, about 0.25 μg/mL, about 0.5 μg/mL, about 1 μg/mL, or about 2 μg/mL hydrocortisone. In some embodiments, the culture medium comprises from about 0.01 μg/mL to about 0.05 μg/mL, from about 0.01 μg/mL to about 0.1 μg/mL, from about 0.01 μg/mL to about 0.5 μg/mL, from about 0.05 μg/mL to about 1 μg/mL, from about 0.05 μg/mL to about 2 μg/mL, from about 0.02 μg/mL to about 0.1 μg/mL, 0.02 μg/mL to about 0.2 μg/mL, from about 0.02 μg/mL to about 0.5 μg/mL, from about 0.02 μg/mL to about 1 μg/mL, from about 0.02 μg/mL to about 2 μg/mL, from about 0.05 μg/mL to about 1 μg/mL, from about 0.05 μg/mL to about 2 μg/mL, from about 0.5 μg/mL to about 1 μg/mL, from about 0.5 μg/mL to about 2 μg/mL, or from about 1 μg/mL to about 2 μg/mL hydrocortisone. In some embodiments, the culture medium comprises from about 0.02 μg/mL to about 0.2 μg/mL hydrocortisone. In some embodiments, the culture medium comprises about 0.2 μg/mL hydrocortisone.

In some embodiments, the culture medium comprises: from about 0.5 ng/ml to about 50 ng/mL VEGF, from about 0.5 ng/mL to about 50 ng/ml EGF, from about 1 ng/mL to about 100 ng/mL bFGF, from about 2 ng/ml to about 200 ng/ml IGF, from about 0.1 μg/mL to about 10 μg/mL Ascorbic acid, or from about 0.02 μg/mL to about 2 μg/mL hydrocortisone. In some embodiments, the culture medium comprises: from about 0.5 ng/ml to about 50 ng/mL VEGF, from about 0.5 ng/ml to about 50 ng/ml EGF, from about 1 ng/mL to about 100 ng/mL bFGF, from about 2 ng/ml to about 200 ng/ml IGF, from about 0.1 μg/mL to about 10 μg/mL Ascorbic acid, and from about 0.02 μg/mL to about 2 μg/mL hydrocortisone. In some embodiments, the culture medium comprises: about 0.5 ng/ml VEGF, about 5 ng/mL EGF, about 10 ng/mL bFGF, about 20 ng/ml IGF, about 1 μg/mL Ascorbic acid, or about 0.2 μg/mL hydrocortisone. In some embodiments, the culture medium comprises: about 0.5 ng/mL VEGF, about 5 ng/mL EGF, about 10 ng/ml bFGF, about 20 ng/ml IGF, about 1 μg/mL Ascorbic acid, and about 0.2 μg/mL hydrocortisone.

In some embodiments of the present disclosure, the culture medium is a xenofree medium, such as Miltenyi StemMACS™ MSC Expansion Media. In some embodiments of the present disclosure, the culture medium is a xenofree medium, such as Miltenyi MSC-Brew GMP Medium.

As shown by Example 1, these components markedly increased the growth rate of the cells, compared to standard media. As further shown by Example 1, this effect could be increased by adding bile acids, such as TCA.

### Cell Culture

Disclosed herein, in certain embodiments, are cell cultures comprising: (a) a human adipose-derived regenerative cell (ADRC) preparation, and (b) cell culture mediums useful in generating human adipose-derived stromal cells (ASCs).

In some embodiments, the ADRC preparation is isolated from a lipoaspirate obtained from a liposuction.

In some embodiments, the ADRC preparation is mechanically isolated or chemically isolated. In some embodiments, the ADRC preparation is treated with collagenase to yield an isolated ADRC preparation.

In some embodiments, the ADRC preparation comprises stromal cells, mesenchymal stem cells (MSCs), endothelial cells, vascular smooth muscle cells, and cells of hematopoietic origin.

Disclosed herein, in certain embodiments, are methods of generating human ASC populations, comprising contacting a preparation of ADRCs with a cell culture medium useful in generating human ASCs. In some embodiments, the methods of generating human ASC populations, comprises: (a) providing (i) an ADRC preparation, and (ii) a cell culture medium useful in generating human ASCs disclosed herein; (b) contacting the ADRC preparation with the culture medium for a time sufficient to allow growth of the MSCs in the ADRC preparation, thereby providing adipose-derived stromal cells (ASCs). In some embodiments, the method further comprises enriching the ASCs from the ADRC preparation.

In some embodiments, the method further comprises enriching regenerative factors or extracellular vesicles released from the ASCs, thereby providing a preparation of factors or extracellular vesicles released from the ASCs.

The compounds of the cell culture mediums can be kept at suitable levels. In some embodiments, the culture medium is exchanged for every 3-4 days. In some embodiments of the present disclosure, the compounds and/or bile acids are replenished by addition directly to the media.

The function of the ASCs are not limited to the type of cell culture, and various ways to culture the cells are contemplated. In some embodiments of the present disclosure, the method is performed using microcarriers as a growth surface. In some embodiments of the present disclosure, the method is performed in a 3-D culture, such as a bioreactor, where the gas composition and pH are continuously monitored and adjusted.

In some embodiments of the present disclosure, the method is performed in a 2-D culture. In some embodiments, the culture system is selected from the group consisting of shakeflask, roller bottle, spinner flask, plate, stirred tank bioreactor, wave reactor, and a fixed bed reactor. In some embodiments, the culture system comprises a culture-compatible surface. In some embodiments, the culture-compatible surface comprises a synthetic substrate, a plastic substrate, a polymer, an extracellular matrix-mimicking coating, or combinations thereof.

In some embodiments, the ADRC preparation is cultured for at least 1 passage, such as at least 2 passages, such as at least 5 passages, such as at least 10 passages. In some embodiments of the present disclosure, the ADRC preparation is cultured for at least 3 passages. In some embodiments of the present disclosure, the ADRC preparation is cultured for at least 5 passages. In some embodiments of the present disclosure, the ADRC preparation is cultured for at least 6 passages. In some embodiments of the present disclosure, the ADRC preparation is cultured for at least 8 passages. In some embodiments of the present disclosure, the ADRC preparation is cultured for at least 10 passages. In some embodiments of the present disclosure, the ADRC preparation is cultured for 3, 4, 5, 6, 7, 8, 9, 10, 11, 12, 13, or 14 passages. In some embodiments of the present disclosure, the ADRC preparation is cultured for 3 passages. In some embodiments of the present disclosure, the ADRC preparation is cultured for 4 passages. In some embodiments of the present disclosure, the ADRC preparation is cultured for 5 passages. In some embodiments of the present disclosure, the ADRC preparation is cultured for 6 passages. In some embodiments of the present disclosure, the ADRC preparation is cultured for 7 passages. In some embodiments of the present disclosure, the ADRC preparation is cultured for 8 passages. In some embodiments of the present disclosure, the ADRC preparation is cultured for 9 passages. In some embodiments of the present disclosure, the ADRC preparation is cultured for 10 passages.

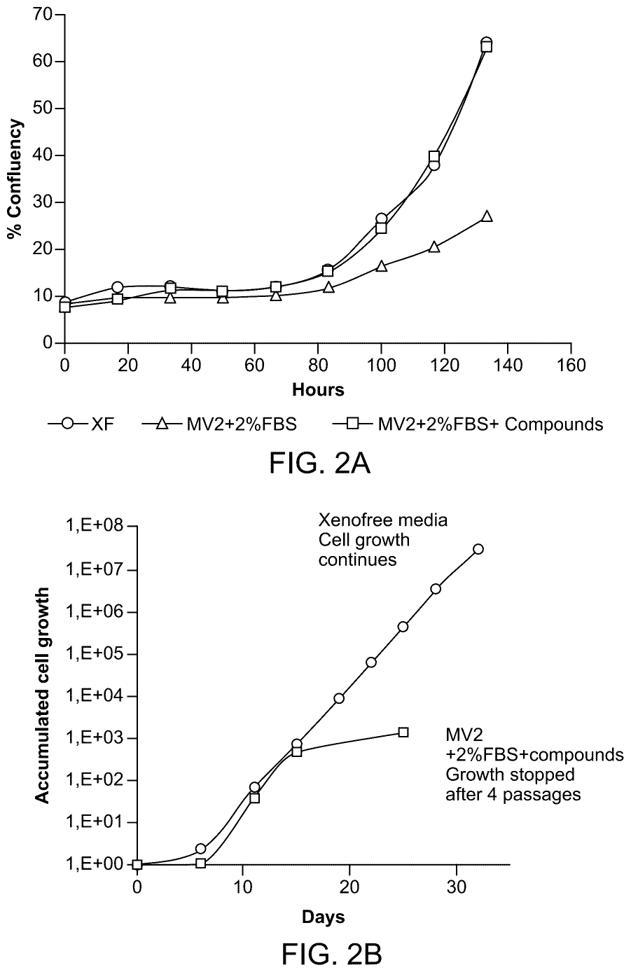

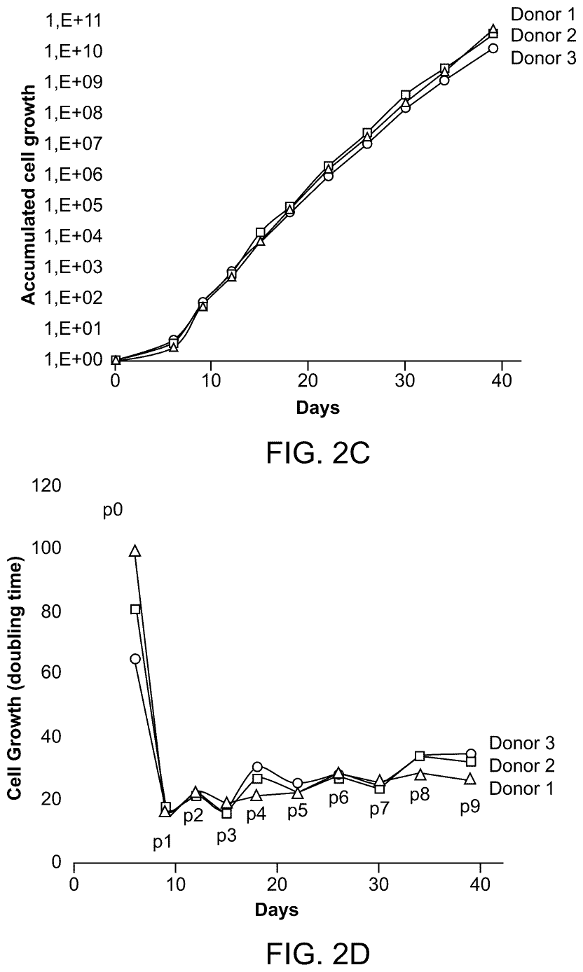

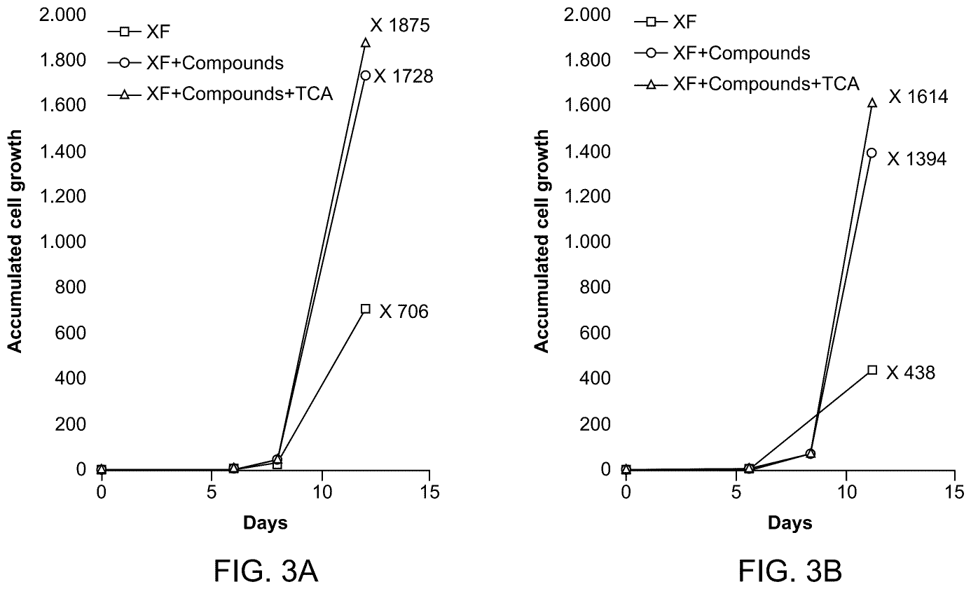

As visualized by FIGS. 2A-3B, the time to reach 70% confluence or the growth potential over time is increased by the method as disclosed herein. The skilled person will however find that the exact time to reach confluence may be highly dependent on other factors as well. The time to reach confluence is furthermore affected by the amount of passages the cells have been grown for. For instance, the initially seeded cells will be slower, than later passaged cells, since the ADRC preparation will comprise debris and cells not capable of attaching to the plastic surfaces. When the ASCs have been grown for at least one passage on a plastic surface, the time for a passage will increase. In some embodiments of the present disclosure, the time to reach 70-80% confluency does not exceed 160 hours, such as does not exceed 150 hours, such as does not exceed 140 hours. In some embodiments of the present disclosure, the time to reach 70-80% confluency in a first seed, i.e. the cells have not been passaged, does not exceed 160 hours, such as does not exceed 150 hours, such as does not exceed 140 hours.

Since a passage, and the time taken to grow into confluence, may be prone to variations, another relevant parameter to employ is the doubling time. For example, 10 passages is more than 30 cumulative population doubling (cPD) and thus around 3.5 doublings (at least) per passage. This can be dependent on the number of cells seeded and the number of days you grow. The doubling time is thus more stable, and can be tightly monitored, as for instance visualized by FIG. 2D. A way of monitoring the doubling time is to count the actual increase in cells in a measured. In some embodiments of the present disclosure, the ADRC preparation is cultured for at least 2, 3, 4, 5, 6, 7, 8, 9, 10, 11, 12, 13, 14, 15, 16, 17, 18, 19, 20, 24, 28, 32, 36, 40, or more than 40 cPDs. In some embodiments of the present disclosure, the ADRC preparation is cultured for about 2, 3, 4, 5, 6, 7, 8, 9, 10, 11, 12, 13, 14, 15, 16, 17, 18, 19, 20, 24, 28, 32, 36, 40, or more than 40 cPDs. In some embodiments of the present disclosure, the ADRC preparation is cultured in a range from about 2 to about 40, from about 2 to about 30, from about 2 to about 25, from about 2 to about 20, from about 2 to about 16, from 2 to about 12, or from 2 to about 10 cPDs. In some embodiments of the present disclosure, the ADRC preparation is cultured in a range from about 4 to about 40, from about 4 to about 30, from about 4 to about 25, from about 4 to about 20, from about 4 to about 16, from 4 to about 12, or from 4 to about 10 cPDs. In some embodiments of the present disclosure, the ADRC preparation is cultured in a range from about 6 to about 40, from about 6 to about 30, from about 6 to about 25, from about 6 to about 20, from about 6 to about 16, from 6 to about 12, or from 6 to about 10 cPDs. In some embodiments of the present disclosure, the ADRC preparation is cultured in a range from about 10 to about 40, from about 10 to about 30, from about 10 to about 25, from about 10 to about 20, from about 10 to about 16, or from 10 to about 12 cPDs. In some embodiments of the present disclosure, the ADRC preparation is cultured for at least 9 cPDs, such as at least 15 cPD, such as at least 30 cPDs. In some embodiments of the present disclosure, the ADRC preparation is cultured for at least 30 cPDs.

In some embodiments, the doubling time for a cell grown by a method as described herein is between 17-35 hours. As seen from FIG. 2A, the first seeding is slower than later passages, and thus preferably, the cells have been passaged at least one time before measuring their doubling time. In the first passage doubling time is high, due to low plating efficiency (FIG. 2D).

When the cells are grown in a 3D culture, it is especially preferred to use the doubling time, since these cells are not passaged.

In some embodiments, the cells are cultured in standard cell culture conditions, i.e. at 37° C., high relative humidity, a standard oxygen level (e.g. 21% in a 5% CO₂ and 95% air mixture), and pH control 7.2 to 7.4.

In some embodiments, the cells are cultured at a temperature from about 27° C. to about 39° C. In some embodiments, the cells are cultured at a temperature from about 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C. or 39° C., or any value or range therein. In some embodiments, the cells are cultured at a temperature from about 35° C. to about 38° C., from about 36° C. to about 39° C., from about 36.5° C. to about 39° C., from about 36.5° C. to about 37.5° C., or from about 36.5° C. to about 38° C. In some embodiments, the cells are cultured at temperature from about 30° C. to about 37° C. In some embodiments, the cells are cultured at a temperature of about 30° C., 30.5° C., 31° C., 31.5° C., 32° C., 32.5° C., 33° C., 33.5° C., 34° C., 34.5° C., 35° C., 35.5° C., 36° C., 36.5° C., or 37° C., or any value or range therein. In some embodiments, the cells are cultured at a temperature from about 32° C. to about 35° C., from about 33° C. to about 36° C., from about 33.5° C. to about 36° C., from about 32.5° C. to about 35.5° C., or from about 34.5° C. to about 37° C.).

In some embodiments, the cells are cultured at a high relative humidity from about 55% to about 100%. In some embodiments, the cells are cultured at a high relative humidity from about 55%, 60%, 65%, 70%, 75%, 80%, 85%, 90%, 95% or 100%, or any value or range therein. In some embodiments, the cells are cultured at a high relative humidity from about 55% to about 95%, from about 60% to about 95%, from about 65% to about 95%, from about 70% to about 95%, from about 75% to about 95%, from about 80% to about 95%, from about 85% to about 95%.

In some embodiments, the cells are cultured at an oxygen level of about 2%, 2.5%, 3.0%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, or 22% oxygen or any value or range therein. In some embodiments, the cells are cultured at an oxygen level from about 2% to about 21.5%, from about 3% to about 21%, from about 4% to about 21%, from about 5% to about 21%, from about 6% to about 21%, from about 7% to about 21%, from about 8% to about 21%, from about 9% to about 21%, or from about 10% to about 21%, from about 11 to about 21%, from about 12% to about 21%, from about 13% to about 21%, from about 14% to about 21%, from about 15% to about 21%, or from about 16% to about 21%, from about 17% to about 21%, from about 18% to about 21%, from about 18% to about 21%, from about 20% to about 21%, from about 20.5% to about 21%, or from about 20.5% to about 21.5% oxygen. In some embodiments, the cells are cultured at an oxygen level from about 2% about 8% oxygen. In some embodiments, the cells are cultured at an oxygen level of about 21% oxygen.

In some embodiments, the cells are cultured at a CO₂ level of about 3%, 3.25%, 3.5%, 3.75%, 4%, 4.25%, 4.5%, 4.75%, 5%, 5.25%, 5.5%, 5.75%, 6%, 6.25%, or 6.5% CO₂ or any value or range therein. In some embodiments, the cells are cultured at a CO₂ level from about 3% to about 4.5%, from about 3.5% to about 5%, from about 4% to about 5.5%, from about 4.5% to about 6%, from about 5% to about 6.5%, from about 4% to about 5%, from about 4.5% to about 5.5%, from about 5% to about 6%, or from about 5.5% to about 6.5% CO₂. In some embodiments, the cells are cultured at a CO₂ level from about 4% to about 6% CO₂. In some embodiments, the cells are cultured at a CO₂ level of about 5% CO₂.

In some embodiments, the cells are cultured at a pH of 6.8, 6.9, 7.0, 7.1, 7.2, 7.3, 7.4, 7.5, or 7.6. In some embodiments, the cells are cultured at a pH of a range from about 6.8 to about 7.6, from about 6.8 to about 7.4, from about 6.8 to about 7.2, from about 6.8 to about 7.0, from about 7.0 to about 7.6, from about 7.0 to about 7.4, from about 7.0 to about 7.2, from about 7.2 to about 7.6, or from about 7.2 to about 7.4. In some embodiments, the cells are cultured at a pH of a range from about 7.2 to about 7.4.

Cells, Vesicles, and Media Obtained from the Method and their Use in Treatment

From the above described methods and cell culture it is possible to obtain ASCs, i.e. MSCs cultured from an ADRC preparation. In some embodiments, it is not necessary to further enrich or enhance the amount of ACSs, before they are used in further applications.

Disclosed herein, in certain embodiments, are mesenchymal stem cells obtained using the cell culture medium and methods described above. In some embodiments, the mesenchymal stem cells differentially express one or more proteins. In some embodiments, the mesenchymal stem cells differentially express one or more proteins as seen in Table 3 and Table 4. In some embodiments, the mesenchymal stem cells express (e.g., have present) or have increased expression (e.g., have upregulated) of the one or more proteins. In some embodiments, the mesenchymal stem cells have no expression (e.g., have absent) or have decreased expression (e.g., downregulated) of the one or more proteins.

In some embodiments, the mesenchymal stem cells differentially express (e.g., have absent, have present, downregulated, or upregulated) the one or more proteins following culture of ADRCs in a first condition as compared to a second condition, third condition, fourth condition, fifth condition, and/or sixth condition. In some embodiments, the first condition is a xenofree media with growth factor cocktail/compounds (EGF (5 ng/ml), bFGF (10 ng/ml), IGF (20 ng/mL), VEGF (0.5 ng/mL), Ascorbic acid 1 μg/mL, hydrocortisone (0.2 μg/mL), and TCA (10 μM)) and culture up to 5 passages. In some embodiments, the second condition is a xenofree media with growth factor cocktail/compounds (EGF (5 ng/mL), bFGF (10 ng/ml), IGF (20 ng/mL), VEGF (0.5 ng/ml), Ascorbic acid 1 μg/mL, hydrocortisone (0.2 μg/mL), and TCA (10 μM)) and culture up to two passages. In some embodiments, the third condition is a xenofree media with growth factor cocktail/compounds (EGF (5 ng/ml), bFGF (10 ng/ml), IGF (20 ng/mL), VEGF (0.5 ng/ml), Ascorbic acid 1 μg/mL, hydrocortisone (0.2 μg/mL), and TCA (10 μM)) and culture of cells to a cell density greater than about 80% confluency (e.g., about 85%, 90%, or 95% confluency). In some embodiments, the fourth condition is a xenofree media with growth factor cocktail/compounds (EGF (5 ng/ml), bFGF (10 ng/ml), IGF (20 ng/mL), VEGF (0.5 ng/ml), Ascorbic acid 1 μg/mL, hydrocortisone (0.2 μg/mL), and TCA (10 μM)) and culture up to 15 passages. In some embodiments, the fifth condition is a xenofree media with growth factor cocktail/compounds (EGF (5 ng/ml), bFGF (10 ng/mL), IGF (20 ng/mL), VEGF (0.5 ng/mL), Ascorbic acid 1 μg/mL, hydrocortisone (0.2 μg/mL), and TCA (10 μM)) and use of a differentiation medium. In some embodiments, the sixth condition is no culture.

In some embodiments, the mesenchymal stem cells have at least about [LOG₂]₃-fold lower expression of the one or more proteins using the first condition as compared to a cell population grown using the second condition, third condition, fourth condition, fifth condition, and/or sixth condition. In some embodiments, the mesenchymal stem cells have at least about [LOG₂]₃, [LOG₂]₄, [LOG₂]₅, [LOG₂]₆, [LOG₂]₇, [LOG₂]₈, [LOG₂]₉, or [LOG₂]₁₀-fold lower expression of the one or more proteins using the first condition as compared to a cell population grown using the second condition, third condition, fourth condition, fifth condition, and/or sixth condition.

In some embodiments, the mesenchymal stem cells have at least about [LOG₂]₃-fold higher expression of the one or more proteins using the first condition as compared to a cell population grown using the second condition, third condition, fourth condition, fifth condition, and/or sixth condition. In some embodiments, the mesenchymal stem cells have at least about [LOG₂]₃, [LOG₂]₄, [LOG₂]₅, [LOG₂]₆, [LOG₂]₇, [LOG₂]₈, [LOG₂]₉, or [LOG₂]₁₀-fold higher expression of the one or more proteins using the first condition as compared to a cell population grown using the second condition, third condition, fourth condition, fifth condition, and/or sixth condition.

In some embodiments, the mesenchymal stem cells: a) express or have increased expression of a protein selected from the group consisting of: ADAM15, ADIRF, AMD1, CDCA8, CDKN2C, CHST11, CXCL5, DKK2, GPALPP1, IGFBP2, LRP11, MEST, MMP1, MT2A, NEO1, NPTX1, PRXL2A, SMOC1, SRGN, ST6GAL2, TNFRSF6B, TPPP3, TRIAP1, ULBP2, VEGFD, CDK1, F5, GINS3, KPNA2, MKI67, NCAPD2, PBK, POLA1, PRG4, SCUBE3, SPDL1, TOP2A, TYMS, UHRF1, and EFHD2 and/or b) have no expression or decreased expression of a protein selected from the group consisting of: AOC3, C2, CFD, CRIP1, F9, IGLV3-19, NTNG1, SERPINB7, SNCA, BGN, CKB, COL12A1, COL14A1, DCN, FBLN1, FCN3, FUCA1, GPLD1, GPNMB, HSPB6, HTRA1, ICAM1, KRT86, LTF, LUM, MX1, POSTN, RAB27B, SERPINB2, SOD2, TGM2, TNC, ALDH2, DPP4, SECTM1, and AKRIC1.

In some embodiments, the mesenchymal stem cells express a protein selected from the group consisting of: ADAM15, ADIRF, AMD1, CDCA8, CDKN2C, CHST11, CXCL5, DKK2, GPALPP1, IGFBP2, LRP11, MEST, MMP1, MT2A, NEO1, NPTX1, PRXL2A, SMOC1, SRGN, ST6GAL2, TNFRSF6B, TPPP3, TRIAP1, ULBP2, and VEGFD. In some embodiments, the mesenchymal stem cells have increased expression of a protein selected from the group consisting of: CDK1, F5, GINS3, KPNA2, MKI67, NCAPD2, PBK, POLA1, PRG4, SCUBE3, SPDL1, TOP2A, TYMS, UHRF1, and EFHD2.

In some embodiments, the mesenchymal stem cells have decreased expression of a protein selected from the group consisting of: BGN, CKB, COL12A1, COL14A1, DCN, FBLN1, FCN3, FUCA1, GPLD1, GPNMB, HSPB6, HTRA1, ICAM1, KRT86, LTF, LUM, MX1, POSTN, RAB27B, SERPINB2, SOD2, TGM2, TNC, ALDH2, DPP4, SECTM1, and AKRIC1. In some embodiments, the mesenchymal stem cells have no expression of a protein selected from the group consisting of: AOC3, C2, CFD, CRIP1, F9, IGLV3-19, NTNG1, SERPINB7, and SNCA.

In some embodiments, the mesenchymal stem cells: a) express or have increased expression of a protein selected from the group consisting of: CXCL5, EFHD2, IGFBP2, MEST, NPTX1, PRG4, SCUBE3, SRGN, ST6GAL2, and TNFRSF6B and/or b) have no expression or decreased expression of a protein selected from the group consisting of: AKRIC1, ALDH2, AOC3, C2, DPP4, GPNMB, IGLV3-19, SECTM1, and SERPINB2. In some embodiments, the mesenchymal stem cells: a) express or have increased expression of SRGN and/or b) have no expression or decreased expression of a protein selected from the group consisting of: AOC3, C2, and IGLV3-19.

In some embodiments, the mesenchymal stem cells: a) express or have increased expression of a protein selected from the group consisting of: ADAM15, AMD1, CDCA8, CDK1, CHST11, CXCL5, DKK2, EFHD2, GINS3, KPNA2, MEST, MKI67, MMP1, NCAPD2, NPTX1, PBK, POLA1, SCUBE3, SMOC1, SPDL1, ST6GAL2, TOP2A, TYMS, and UHRF1 and/or b) have no expression or decreased expression of a protein selected from the group consisting of: AKRIC1, ALDH2, CFD, COL14A1, CRIP1, DCN, DPP4, FBLN1, FCN3, FUCA1, GPLD1, GPNMB, HSPB6, KRT86, LUM, MX1, RAB27B, SECTM1, SERPINB2, SOD2, and TGM2. In some embodiments, the mesenchymal stem cells: a) express or have expresses or has increased expression of a protein selected from the group consisting of: ADAM15, MMP1, and SMOC1 and/or b) have no expression or decreased expression of a protein selected from the group consisting of: CFD, CRIP1, FCN3, and KRT86.

In some embodiments, the mesenchymal stem cells: a) express or have increased expression of a protein selected from the group consisting of: AMD1, CDCA8, CDK1, CDKN2C, CHST11, DKK2, EFHD2, F5, GINS3, GPALPP1, IGFBP2, KPNA2, LRP11, MKI67, NCAPD2, NEO1, PBK, POLA1, PRXL2A, SPDL1, ST6GAL2, TNFRSF6B, TOP2A, TPPP3, TYMS, UHRF1, ULBP2, and VEGFD and/or have no expression or decreased expression of a protein selected from the group consisting of: AKRIC1, ALDH2, CKB, COL14A1, DPP4, F9, FUCA1, GPLD1, GPNMB, HSPB6, ICAM1, LTF, MX1, NTNG1, RAB27B, SECTM1, SERPINB2, SERPINB7, SNCA, SOD2, and TGM2. In some embodiments, the mesenchymal stem cells: a) express or have increased expression of a protein selected from the group consisting of: CDKN2C, F5, LRP11, NEO1, PRXL2A, TPPP3, ULBP2, and VEGFD and/or b) have no expression or decreased expression of a protein selected from the group consisting of: CKB, F9, ICAM1, LTF, NTNG1, SERPINB7, and SNCA.

In some embodiments, the mesenchymal stem cells: a) express or have increased expression of a protein selected from the group consisting of: ADIRF, AMD1, CDCA8, CXCL5, EFHD2, GINS3, GPALPP1, IGFBP2, MT2A, POLA1, PRG4, SCUBE3, SPDL1, ST6GAL2, TNFRSF6B, and TRIAP1 and/or b) have no expression or decreased expression of a protein selected from the group consisting of: AKRIC1, ALDH2, BGN, COL12A1, COL14A1, DCN, DPP4, FBLN1, GPLD1, GPNMB, HTRA1, LUM, POSTN, RAB27B, SECTM1, SERPINB2, and TNC. In some embodiments, the mesenchymal stem cells: a) express or have increased expression of a protein selected from the group consisting of: ADIRF, MT2A, and TRIAP1 and/or b) have no expression or decreased expression of a protein selected from the group consisting of: BGN, COL12A1, HTRA1, POSTN, and TNC.

In some embodiments, the mesenchymal stem cells express or have increased expression of a protein selected from the group consisting of: MEST and NPTX1.

In some embodiments, the mesenchymal stem cells express or have increased expression of PRG4.

In some embodiments, the mesenchymal stem cells express or have increased expression of a protein selected from the group consisting of: DKK2, CHST11, CDK1, KPNA2, MKI67, NCAPD2, PBK, TOP2A, TYMS, and UHRF1 and/or b) has no expression or decreased expression of a protein selected from the group consisting of: FUCA1, HSPB6, MX1, SOD2, and TGM2.

In some embodiments, the mesenchymal stem cells have no expression or decreased expression of a protein selected from the group consisting of: DCN, FBLN1, and LUM.

In some embodiments, the mesenchymal stem cells express or have increased expression of GPALPP1.

In some embodiments, the mesenchymal stem cells express or have increased expression of a protein selected from the group consisting of: CXCL5 and SCUBE3.

In some embodiments, the mesenchymal stem cells express or have increased expression of a protein selected from the group consisting of: IGFBP2 and TNFRSF6B.

In some embodiments, the mesenchymal stem cells express or have increased expression of a protein selected from the group consisting of: AMD1, CDCA8, GINS3, POLA1, and SPDL1 and/or b) has no expression or decreased expression of a protein selected from the group consisting of: COL14A1, GPLD1, and RAB27B.

In some embodiments, the mesenchymal stem cells express or have increased expression of a protein selected from the group consisting of: ST6GAL2 and EFHD2 and/or b) has no expression or decreased expression of a protein selected from the group consisting of: GPNMB, SERPINB2, ALDH2, DPP4, SECTM1, and AKRIC1.

In some embodiments, the mesenchymal stem cells express a cell surface protein or a secreted protein. In some embodiments, the mesenchymal stem cells: a) express or have increased expression of a protein selected from the group consisting of: CXCL5, DKK2, IGFBP2, MMP1, NEO1, NPTX1, SMOC1, SRGN, TNFRSF6B, ULBP2, VEGFD, F5, and SCUBE3 and/or b) have no expression or decreased expression of a protein selected from the group consisting of: AOC3, C2, CFD, F9, IGLV3-19, NTNG1, SNCA, BGN, CKB, COL12A1, COL14A1, DCN, FBLN1, FCN3, GPLD1, HSPB6, HTRA1, LTF, LUM, POSTN, SERPINB2, TGM2, TNC.

In some embodiments, the mesenchymal stem cells: a) express or have increased expression of a protein selected from the group consisting of: CXCL5, IGFBP2, NPTX1, SCUBE3, SRGN, and TNFRSF6B and/or b) has no expression or decreased expression of a protein selected from the group consisting of: AOC3, C2, IGLV3-19, and SERPINB2. In some embodiments, the mesenchymal stem cells: a) express or have increased expression of SRGN and/or b) have no expression or decreased expression of a protein selected from the group consisting of: AOC3, C2, and IGLV3-19.

In some embodiments, the mesenchymal stem cells: a) express or have increased expression of a protein selected from the group consisting of: CXCL5, DKK2, MMP1, NPTX1, SCUBE3, and SMOC1 and/or b) has no expression or decreased expression of a protein selected from the group consisting of: CFD, COL14A1, DCN, FBLN1, FCN3, GPLD1, HSPB6, LUM, SERPINB2, and TGM2. In some embodiments, the mesenchymal stem cells: a) express or have increased expression of a protein selected from the group consisting of: MMP1 and SMOC1 and/or b) has no expression or decreased expression of a protein selected from the group consisting of: CFD and FCN3.

In some embodiments, the mesenchymal stem cells: a) express or have increased expression of a protein selected from the group consisting of: DKK2, F5, IGFBP2, NEO1, TNFRSF6B, ULBP2, and VEGFD and/or b) have no expression or decreased expression of a protein selected from the group consisting of: CKB, COL14A1, F9, GPLD1, HSPB6, LTF, NTNG1, SERPINB2, SNCA, and TGM2. In some embodiments, the mesenchymal stem cells: a) express or have increased expression of a protein selected from the group consisting of: F5, NEO1, ULBP2, and VEGFD and/or b) have no expression or decreased expression of a protein selected from the group consisting of: CKB, F9, LTF, NTNG1, and SNCA.

In some embodiments, the mesenchymal stem cells: a) express or have increased expression of a protein selected from the group consisting of: CXCL5, IGFBP2, SCUBE3, and TNFRSF6B and/or b) has no expression or decreased expression of a protein selected from the group consisting of: BGN, COL12A1, COL14A1, DCN, FBLN1, GPLD1, HTRA1, LUM, POSTN, SERPINB2, and TNC. In some embodiments, the mesenchymal stem cells have no expression or decreased expression of a protein selected from the group consisting of: BGN, COL12A1, HTRA1, POSTN, and TNC.

In some embodiments, the mesenchymal stem cells express or have increased expression of NPTX1.

In some embodiments, the mesenchymal stem cells: a) express or have increased expression of DKK2 and/or b) have no expression or decreased expression of a protein selected from the group consisting of: HSPB6 and TGM2.

In some embodiments, the mesenchymal stem cells have no expression or decreased expression of a protein selected from the group consisting of: DCN, FBLN1, and LUM.

In some embodiments, the mesenchymal stem cells express or have increased expression of a protein selected from the group consisting of: CXCL5 and SCUBE3.

In some embodiments, the mesenchymal stem cells express or have increased expression of a protein selected from the group consisting of: IGFBP2 and TNFRSF6B.

In some embodiments, the mesenchymal stem cells have no expression or decreased expression of a protein selected from the group consisting of: COL14A1 and GPLD1.

In some embodiments, the mesenchymal stem cells have no expression or decreased expression of SERPINB2.

Disclosed herein, in certain embodiments, are mesenchymal stem cells expressing increased expression and/or activity of angiogenic factors, neurogenic factors, or both angiogenic factors and neurogenic factors as compared to uncultured cells or cells cultured in a culture medium that does not comprise VEGF, EGF, bFGF, IGF, Ascorbic acid, and hydrocortisone or any functional fragments, variants, or homologous proteins of VEGF, EGF, bFGF, or IGF.

Disclosed herein, in certain embodiments, are ASC populations comprising more than about 95% mesenchymal stem cells expressing increased expression and/or activity of angiogenic factors, neurogenic factors, or both angiogenic factors and neurogenic factors In some embodiments, the ASC population does not comprise stromal cells, endothelial cells, smooth muscle cells, or cells of hematopoietic origin. In some embodiments, the ASC population do not express adipose markers.

In some embodiments, the cell culture medium comprises one or more compounds selected from the group consisting of VEGF, EGF, bFGF, IGF, Ascorbic acid, and hydrocortisone or any functional fragments, variants, or homologous proteins of VEGF, EGF, bFGF, or IGF. In some embodiments, the cell culture medium further comprises one or more bile acids. In some embodiments, the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA). In some embodiments, the bile acid is taurocholic acid (TCA). In some embodiments, the culture medium comprises at least VEGF. In some embodiments, the culture medium comprises VEGF, EGF, bFGF, IGF, Ascorbic acid, and hydrocortisone or any functional fragments, variants, or homologous proteins of VEGF, EGF, bFGF, or IGF.

In some embodiments, the culture medium comprises: from about 0.5 ng/ml to about 50 ng/mL VEGF, from about 0.5 ng/ml to about 50 ng/mL EGF, from about 1 ng/mL to about 100 ng/mL bFGF, from about 2 ng/ml to about 200 ng/ml IGF, from about 0.1 μg/mL to about 10 μg/mL Ascorbic acid, or from about 0.02 μg/mL to about 2 μg/mL hydrocortisone. In some embodiments, the culture medium comprises: from about 0.5 ng/ml to about 50 ng/mL VEGF, from about 0.5 ng/ml to about 50 ng/mL EGF, from about 1 ng/ml to about 100 ng/mL bFGF, from about 2 ng/ml to about 200 ng/ml IGF, from about 0.1 μg/mL to about 10 μg/mL Ascorbic acid, and from about 0.02 μg/mL to about 2 μg/mL hydrocortisone. In some embodiments, the culture medium comprises: about 0.5 ng/ml VEGF, about 5 ng/mL EGF, about 10 ng/mL bFGF, about 20 ng/ml IGF, about 1 μg/mL Ascorbic acid, or about 0.2 μg/mL hydrocortisone. In some embodiments, the culture medium comprises: about 0.5 ng/mL VEGF, about 5 ng/mL EGF, about 10 ng/ml bFGF, about 20 ng/ml IGF, about 1 μg/mL Ascorbic acid, and about 0.2 μg/mL hydrocortisone. In some embodiments, the culture medium is a xenofree medium, optionally wherein the xenofree medium is selected from the group consisting of Miltenyi StemMACS™ MSC Expansion Media and Miltenyi MSC-Brew GMP Medium.

An aspect of the present disclosure relates to a preparation of ASCs, having been in contact with a cell culture medium comprising one or more compounds selected from the group consisting of VEGF, EGF, bFGF, IGF, Ascorbic acid, and hydrocortisone or any functional fragments, variants, or homologous proteins of VEGF, EGF, bFGF, or IGF and optionally one or more bile acids such as taurocholic acid (TCA).

From the methods disclosed it is furthermore possible to obtain vesicles and conditioned media. Since the ACSs will secrete factors and extracellular vesicles, it is also possible to either use the medium, wherein the cells have grown, or enrich the factors or extracellular vesicles. Thus, in further aspects, the disclosure also relates to a preparation of factors or extracellular vesicles released from ASCs obtained by or obtainable by the method as disclosed herein.

An aspect of the present disclosure relates to a container comprising the ASC preparation as described herein. An aspect of the present disclosure relates to a container comprising the preparation of factors or extracellular vesicles as described herein. An aspect of the present disclosure relates to a container comprising the conditioned medium as described herein.

An aspect of the present disclosure relates to the ASC preparation as described herein for use as a medicament. An aspect of the present disclosure relates to the preparation of factors or extracellular vesicles as described herein for use as a medicament. An aspect of the present disclosure relates to the conditioned medium as described herein for use as a medicament. In some embodiments, the ASC preparation, factors, extracellular vesicles, conditioned medium, or combinations thereof are used to treat a disease or disorder requiring angiogenic and/or neurogenic repair (e.g., erectile dysfunction). In some embodiments, the disease or disorder is characterized by insufficient or abnormal vessel growth or postoperative damage to vessels and/or nerves resulting in reduced blood flow and hypoxia or reduced neuronal activity.

Erectile dysfunction (ED), also known as impotence, is a type of sexual dysfunction characterized by the inability to develop or maintain an erection of the penis during sexual activity. Prostate cancer is the most prevalent cancer in men globally. The primary treatment method is surgical prostatectomy. However, this procedure can result in post-operative erectile dysfunction, affecting 14-90% of men depending on the surgical approach. Considering the limitations and dropout rates associated with current medical treatments, stem cell therapy emerges as a promising treatment option. In some embodiments, a single dose of autologous ungrown ARDCs can effectively alleviate the condition in over 50% of the treated men. Another group of patients experiencing erectile dysfunction can be diabetic patients.

An aspect of the present disclosure relates to the ASC preparation as described herein for use in the treatment of erectile dysfunction. An aspect of the present disclosure relates to the preparation of factors or extracellular vesicles as described herein for use in the treatment of erectile dysfunction. An aspect of the present disclosure relates to the conditioned medium as described herein for use in the treatment of erectile dysfunction.

In some embodiments of the present disclosure, the subject experiencing erectile dysfunction.

The methods, cells and treatments as described herein can be used for both allogenic and autologous treatment. In a typical setup, a preparation of cells will be prepared that can be used for treatment in several subjects, thus the treatments being allogenic treatments. However, in specific cases it may also be occur that the subject receiving the ASCs has also provided the ADRC preparation. In some embodiments of the present disclosure, the treatment is an autologous treatment.

It should be noted that embodiments and features described in the context of one of the aspects of the present disclosure also apply to the other aspects of the disclosure.

### EXAMPLES

The disclosure will now be described in further details in the following non-limiting examples.

### Example 1—Optimizing Culture Conditions for MSCs

This Example shows improved growth of ADRCs using a composition of compounds according to an aspect of the disclosure. Additionally, the Example shows this growth can be improved even further by the addition of bile acids, by showing improved growth using TCA.

For the exploitation of the angiogenic and/or neurogenic potential of MSCs, ADRCs were propagated in vitro, and culture conditions for MSCs determined, while preserving their angiogenic and/or neurogenic properties.

### Methods

### ADRC Isolation

Water-jet assisted liposuction was performed on human donors. Liposuctions were performed at private hospitals, who was in charge of the clinical procedure. The removed lipoaspirate was kept in the sealed tissue collector bag and transferred to the laboratories. Liposuction material was transferred directly from the tissue collector bag into a 1 L sterile plastic funnel under sterile conditions. Liposuction material was washed 5 times with Ringers-lactate buffer. Collagenase NB5 was added to a final concentration of 2.5 WU/mL and digestion was performed in a 37 C incubator on a rocking platform. The digestion was stopped by addition of xenofree culture media followed by centrifugation for 5 min at 300 g. Following resuspension of the cell pellet, cells were filtered first through a 100 μm filter and then a 30 μM filter. The isolated ADRCs were cryopreserved in Cryostor CS10 and kept in a liquid nitrogen tank.

### Cell Culture

Cryopreserved ADRCs were thawed in 37° C. water bath and diluted 10× in xenofree culture media, centrifuged at 400 g for 10 minutes and resuspended in media with or without additional stimuli for culture. Media was exchanged every 3-4 days. Upon 70-80% confluency cells were passaged using TrypLE express. At each passage live cell numbers were determined. Growth curves were calculated by normalizing to fold increases from 1 cell.

Medias Used in these Studies:
DMEM with addition of 10% FBS and 1% P/S,
Promocell MV2 growth cell medium with 2% FBS,
Miltenyi StemMACS™ MSC Expansion Media (xenofree media with proprietary growth factor cocktail),
Growth factor cocktail/compounds (EGF (5 ng/mL), bFGF (10 ng/mL), IGF (20 ng/mL), VEGF (0.5 ng/ml), Ascorbic acid (1 μg/mL), hydrocortisone (0.2 μg/mL)).

### Results

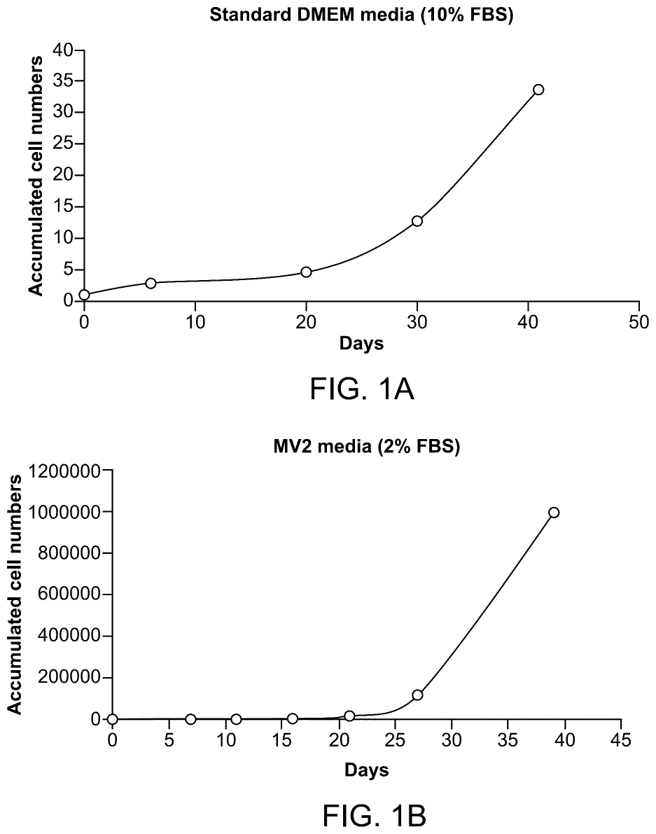

A commonly used media for propagating ADRCs is the standard medium DMEM with 10% FBS and P/S. Growth in such a standard medium with growth in a commercially available cell medium Promocell MV2 with 2% FBS was compared. Surprisingly, the growth of ADRC were 30-fold higher in the MV2 media over a growth period of 40 days (FIGS. 1A-1B).

Next, growth in the MV2 medium with growth in a xenofree MSC medium was compared. The MV2 medium was tested as a basal medium with 2% FBS and with or without addition of MV2 growth factors (FIG. 2A). Initial growth of ADRCs was supported better by addition of the growth factors from the MV2 media. FIG. 2A furthermore shows how the growth in the initial seeding is slow, since the ADRC preparation will comprise debris and cells not capable of attaching to the plastic surfaces. Growth in xenofree and MV2 media was comparable up to 15 days in culture. After this point, growth in MV2 media stopped, whereas the xenofree media supported cell growth up to at least 30 days (>30 cPDs) (FIG. 2B).

The growth in xenofree media was repeated with 3 independent donors (FIG. 2C). The doubling time for each of the passages of the cells grown in xenofree media of FIG. 2C was calculated and plotted (FIG. 2D).

As the MV2 growth factor cocktail potentiated growth in the 2% FBS media, it was determined whether it would also potentiate growth in the xenofree MSC media. Despite the high growth rate in the xenofree media, adding the growth factor cocktail potentiated growth with 2-3-fold over 12 days (FIG. 3A). Further addition of the bile acid taurocholic acid (TCA) to the xenofree media with growth factors increased growth over 3 passages with 8.5 and 15.8% in two independent experiments with cells from two different donors (FIGS. 3A-3B).

### Conclusion

Improved growth of MSCs using a composition of growth factors was determined. The growth factor cocktail improves MSC growth in standard media as well as in xenofree media optimized for MSC growth. Additionally, this growth was improved even further by the addition of bile acids, by showing improved growth in the presence of TCA.

### Example 2—Cultured Cells Express Relevant Cell Markers

This Example describes how the culture conditions affect the identity of the cells and shows cells cultured in xenofree media with addition of compounds and TCA for 3 passages express standard MSC markers and have very low expression of endothelial cell markers and the immunogenicity marker HLA-DR, demonstrating production of a MSC-like cells from the improved culture conditions.

### Methods

Cells were cultured as described in Example 1 in xenofree media, in xenofree media with addition of compounds or in xenofree media with addition of compounds and TCA for up to 3 passages. Upon passaging in passage 1 and 3, cells were frozen in cryopreservation media. For measurement of surface markers, cells were thawed in 10 mL xenofree media, washed in running buffer (containing PBS, stabilizer, EDTA, and sodium azide), and stained with surface antibodies for 10 minutes at 4° C. Cells were analyzed on a flow cytometer.

### Results and Conclusion

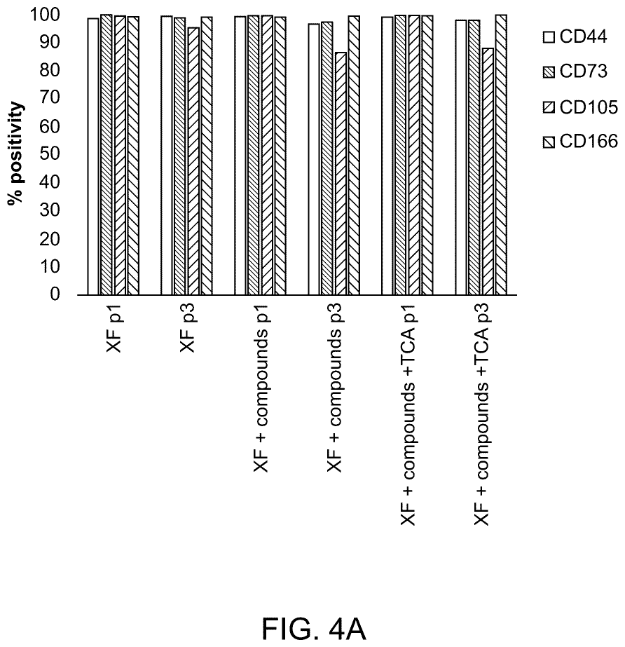

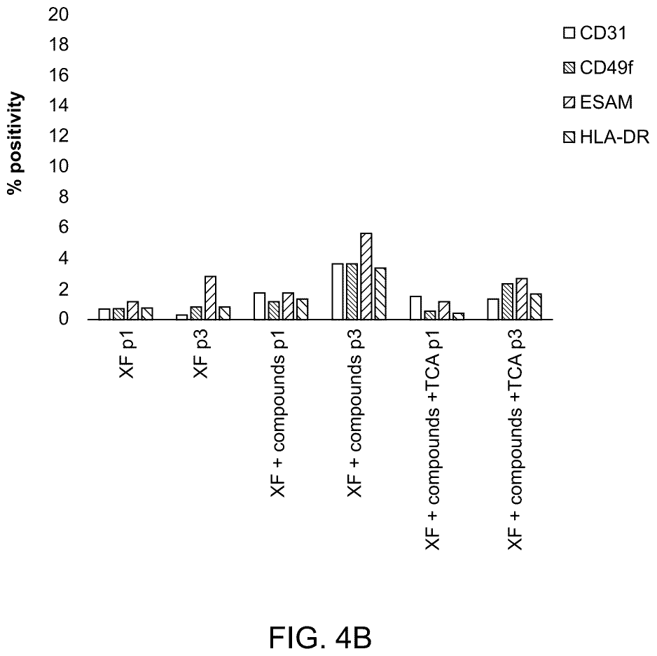

Cells cultured in xenofree media with addition of growth factor cocktail and TCA for 3 passages express standard MSC markers (FIG. 4A) and have very low expression of endothelial cell markers and the immunogenicity marker HLA-DR (FIG. 4B). These expression levels are similar to expression levels on cells cultured in xenofree media without growth factor cocktail.

This Example shows production of a relevant cell type from the improved culture conditions.

### Example 3—Study of Angiogenic Properties

This Example identified the angiogenic properties of cells cultured in the optimized conditions and shows the cells obtained from the optimized culture conditions has maintained or even improved their angiogenic properties, thus showing that cells can be used to treat diseases requiring angiogenic repair. Additionally, the Example also shows that media or factors released in the media from these cells mediate the angiogenic effect.

### Methods

### Scratch Assay

Cryopreserved cells passaged in the growth experiments were thawed as described under cell culture. For each condition 20,000 cells were seeded in a 96-well plate in triplicates. Cells were allowed to attach and 24 hours later the media was changed to starvation media (with no growth factors). After 24 hours this conditioned media containing secreted factors from the ASCs was filtered through a cell filter plate with 3 μM pore size. In parallel a 96-well plate with endothelial cells (HMEC-1) was seeded and starved for 24 hours. A vertical scratch was performed in the cell layer using BioTek AutoScratch. Cell debris was washed off with two washes with PBS. The conditioned media from the ASCs was transferred to the scratched HMEC-1 cells plate. Image analysis was used to calculate the area of the scratch at timepoint 0 hours and after 18 hours. Wound healing was calculated as percentage wound closure/healing.

### Aortic Ring Assay—I

10-12-week-old C57BL6/J male mice were euthanized, and aorta was carefully extracted. Each aorta was cleaned for excess connective tissue under a microscope to ensure complete clean material. After aortas were extracted, they were flushed out in the lumen with cold Heparin and cold starvation media. Cleaned aortas were kept on starvation media at 37 degrees, 5% CO2. First, cells should be seeded in the inserts a day before putting them in contact with the aortic rings. Therefore, the inserts were wet with starvation media for at least 1 h. Afterwards, cells were seeded in the insert in their respective growth media. Next day, aortas were cut into small pieces and embedded in Geltrex. A drop of diluted Geltrex was added to each well. Immediately after, the plate was transferred to an incubator to solidate. Afterwards, one ring per well was added to each well with a tweezer and cover with another drop of Geltrex. Immediately after, the plate was transferred to an incubator to solidate. In the meantime, cells in the inserts were washed carefully two times with warm starvation media/insert. When the rings embedded in Geltrex are solidified, warm starvation media/well with 0.5% FBS was added. Afterwards, cells in the inserts are located on top of the rings and the inserts filled with warm starvation media/well with 0.5% FBS. Cells are cultured together with the rings. Then, inserts are removed from the plate and stained in an empty plate with Calcium-AM in warm HBSS. Subsequently, the inserts are imaged to evaluate the confluency and status of the cells in the inserts. Sprouts from aortic rings are imaged by microscope with phase contrast to detect the complexity of the sprouts. Images were analyzed by Image J-Fiji analyzer to quantify mean area covered by the sprouts.

### Aortic Ring Assay—II

Another version of the above introduced assay was further developed. This assay is also used to investigate if factors secreted by ASCs in culture will stimulate angiogenesis from pieces of aortas. 7-9-weeks-old C57BL6/J male mice were euthanized, and aorta was carefully extracted. Each aorta was cleaned for excess connective tissue under a microscope to ensure complete clean material. After aortas were extracted, they were flushed out in the lumen with cold Heparin and cold media without growth factors (starvation media). Cleaned aortas were kept overnight in starvation media at 37 degrees, 5% CO2. Next day, aortas were cut into rings of 0.5 mm length and embedded in Geltrex diluted with starvation media (7.5 mg/mL final protein concentration). The plates used for the assay was “integrated discrete multiple organ co-culture” (IdMOC) technology, with 6 wells sharing the same media in a unit called a chamber, the plates thus allow for testing of paracrine signaling. The plates used for these experiments had 12 chambers, thus a total of 96 wells. 3 of 6 wells in each chamber was filled with Geltrex and aortic rings. Immediately after, the plate was transferred to an incubator for the Geltrex to solidate. After Geltrex solidification, the remaining 3 wells was filled with starvation media and incubated overnight at 37 degrees, 5% CO2. Next day, the 3 wells with media were emptied, and 3.500 ASCs per well were seeded. Therefore, each chamber will have 3 aortic rings (from 3 different mice) and either 3 wells of ASCs, or 3 empty wells, acting as controls. The individual ASC populations have been subjected to different treatments before initiation of the aortic ring assay. For instance, treatment with xenofree media, and compounds as described herein. Afterwards, ASCs were allowed to attach to the plate for 5 hours. Then, media was removed and the chambers filled with media, to bring individual wells into fluid contact for the first time. All chambers with cells were supplemented with 0.5% FBS starvation media. There were no cells in the negative- or positive control chambers, just aortic rings with 0.5% or 2% FBS in starvation media.

Cells were cultured together with the rings for 5 days. Subsequently, sprouts from aortic rings were imaged by microscope with phase contrast to detect the complexity of the sprouts. Images were analyzed by Image J to quantify mean area covered by the sprouts.

### Matrigel Plug/In Vivo Angiogenesis

10-week-old C57BL6/J female mice were injected subcutaneously into the flank with 500 μl ice-cold Geltrex at a final concentration of 10 mg/mL. The solution and instruments need to be kept on ice at time of injection, as the growth-factor reduced LDEV-free Geltrex (a basement membrane matrix solution produced by murine Engelbreth-Holm-Swarm (EHS) tumors) solidifies at body temperature. Geltrex were supplemented with various grown ADRCs or ASCs at a concentration of 2.5E6 cells/mL or DMEM as a negative control. After 10 days, the animals were euthanized and Geltrex plugs were removed carefully. The plugs were digested for 2 h at 37° C. and 500 rpm shaking in an enzymatic digestive mixture consisting of 3000 U/ml Collagenase and 1000 U/ml Hyaluronidase, and 2667 U/ml DNase dissolved in low glucose DMEM. Following digestion, cells were filtered using a 40 μm nylon mesh. Cells were washed in FACS buffer and stained with murine CD31-PE and murine CD45-VioGreen antibodies to be able to distinguish endothelial cells from the hematopoietic population. Samples were analyzed by flow cytometry.

### Results

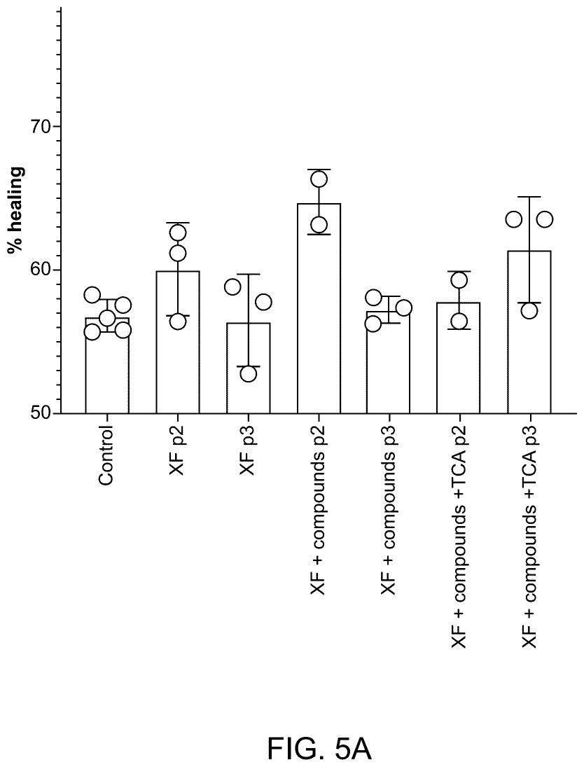

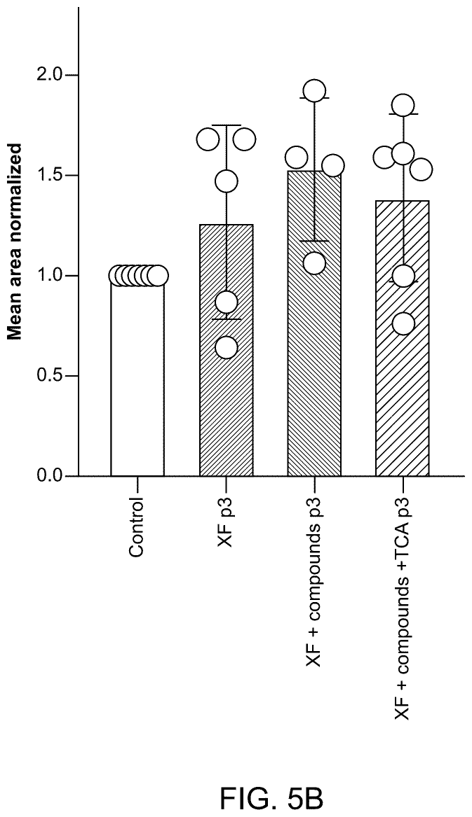

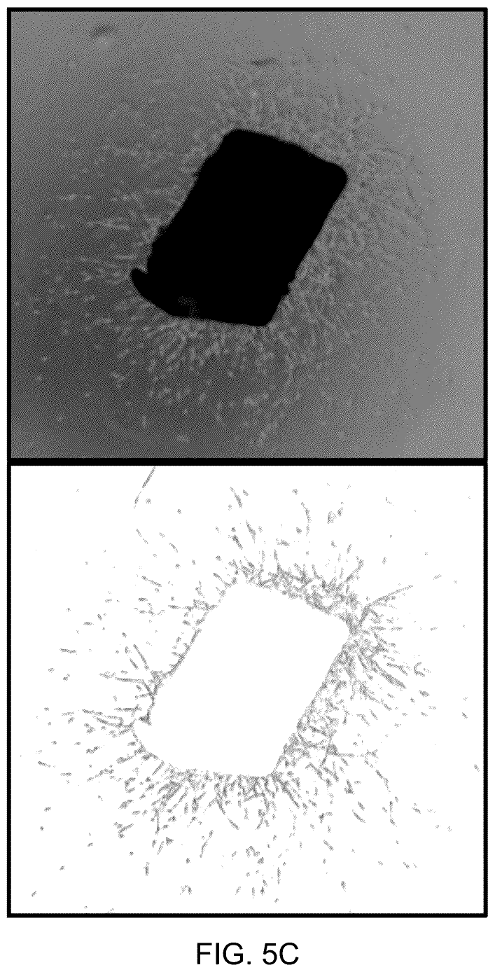

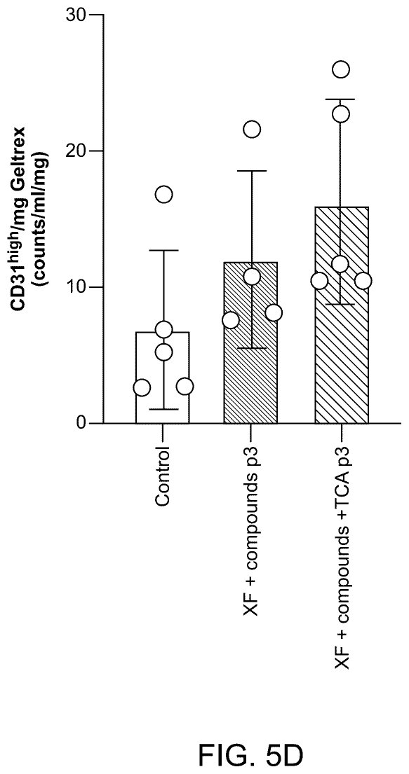

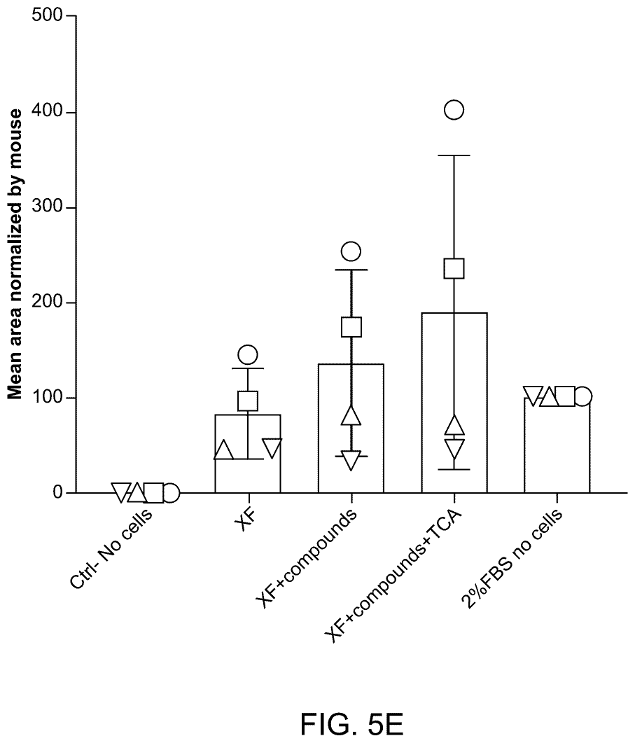

The angiogenic properties of ASCs propagated in xenofree medium with the growth factor cocktail was investigated in an in vitro wound healing, ex vivo aortic ring assays and in vivo Matrigel plug assays (FIGS. 5A-5E). In in vitro wound healing, cultured MSCs showed similar effect after culturing with the 3 different stimuli, whereas in ex vivo and in vivo angiogenesis assays a tendency towards improvement of function was observed with addition of growth factors and TCA (FIGS. 5B and 5D).

### Conclusion

The ASCs obtained from the optimized culture conditions have maintained or even improved their angiogenic properties, thus showing that cells can be used to treat diseases requiring angiogenic repair. In addition, as the cells are not in contact with each other these data show that the angiogenic effect is mediated by factors secreted to the growth media.

### Example 4—Neurite Growth Stimulation by ASCs

This Example describes that factors released by ASCs stimulate neurite growth.

### Methods

Conditioned Media: Cryopreserved ASC preparations, obtained by culturing cells in xenofree media in the presence of compounds and taurocholic acid for 5 passages, were thawed and washed in xenofree media. Cells were cultured for 24 hours in xenofree media plus compounds plus taurocholic acid similar to Example 1, specifically xenofree media with growth factor cocktail/compounds EGF (5 ng/mL), bFGF (10 ng/mL), IGF (20 ng/ml), VEGF (0.5 ng/ml), Ascorbic acid (1 μg/mL), hydrocortisone (0.2 μg/mL), and TCA (10 μM). Media was removed and cells washed thoroughly. Xenofree media without any supplements was added as starvation media and cells were cultured for approximately 22 hours. Media was harvested and frozen. Control media was generated in the same process, but without presence of cells in the culture.

5 to 7 week old C57BL6/J male mice were euthanized and immediately perfused with PBS, pre-warmed to 37° C., by perforating the right heart ventricle and injection of PBS into the left ventricle. The spine was extracted; surrounding connective tissue, fat, and muscle were removed. The spine was cut sagittal into 2 pieces and dorsal root ganglia were removed and stored in 15 mL ice cold DMEM before enzymatic digestion. Enzymatic digestion was performed in PBS containing 2 mg/mL collagenase NB5 and incubation at 37° C. using on an orbital shaker for 30 minutes. After incubation, neurons were separated from tissue by trituration using a 1 mL pipette tip and 10 mL DMEM containing 10% FBS was added. Collagenase solution was washed off via centrifugation and neurons were resuspended in DMEM containing 4 mM Glutamine. Neurons were seeded on ibidi 18-well μ-slide precoated with 50 μg Poly-D-Lysin and lug Laminin. Neurons were either cultured in 50% DMEM containing 4 mM Glutamine and 50% control media, or cultured in 50% DMEM containing 4 mM Glutamine and 50% conditioned media harvested after 24 hours from ASC preparation. The neuron cultures were stimulated in the presence of 0.5% FBS or left unstimulated. After approximately 24 hours, the culture media was removed, and neuron cultures were fixed in 4% paraformaldehyde. The fixed cultures were stained with rabbit beta-III-tubulin antibody and anti-rabbit Alexa488 secondary antibody as well as with Hoechst 33342 for staining of nuclei. The stained cells were then acquired with an automated confocal fluorescence microscope and images analyzed. The total neurite length was calculated as the sum of neurite lengths of each neuron within a culture well and reported in μm.

### Results

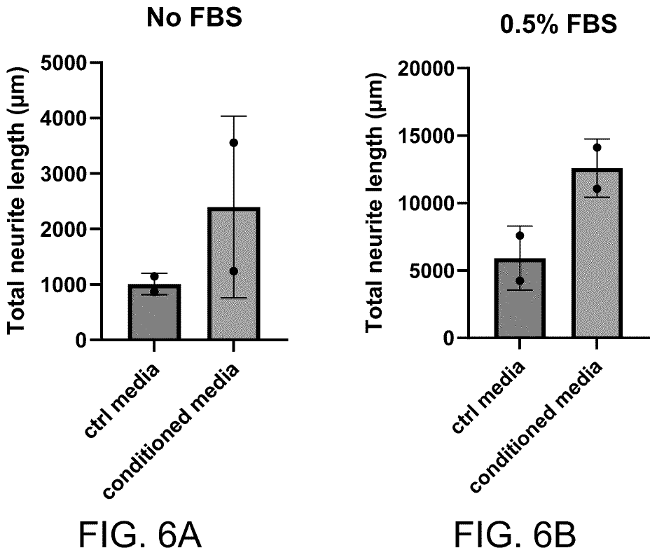

At low stimulation with 0.5% FBS, the total neurite lengths in neuron cultures with conditioned media harvested from ASC cultures was longer compared to neuron cultures with control media. In the absence of any stimulation with FBS, a similar trend is observed. Data is seen in FIGS. 6A-6B.

### Conclusion

Paracrine factors released from ASCs stimulate outgrowth of neurites from neurons, indicating a neurogenic effect of ASC preparations from cultures obtained with xenofree media plus compounds plus TCA.

### Example 5—Study of the Transcriptome

This Example describes gene expression results of cells cultured using methods described herein.

### Methods

RNA was isolated from cryopreserved cells from growth experiments (3 donors, passage 5) for transcriptome analysis of ASCs expanded in xenofree media with growth factor cocktail/compounds (EGF (5 ng/ml), bFGF (10 ng/ml), IGF (20 ng/ml), VEGF (0.5 ng/ml), Ascorbic acid 1 μg/mL, hydrocortisone (0.2 μg/mL), TCA (10 μM)). Differential expression of genes in cells from the two different growth conditions was analyzed based on Next Generation Sequencing. A threshold of 2-fold differential expression and p-value of 0.5 in the Wald-test was applied for identification of regulated genes. The functional context of the regulated genes was studied by comparison of the differentially expressed genes towards gene ontology database.

### Results

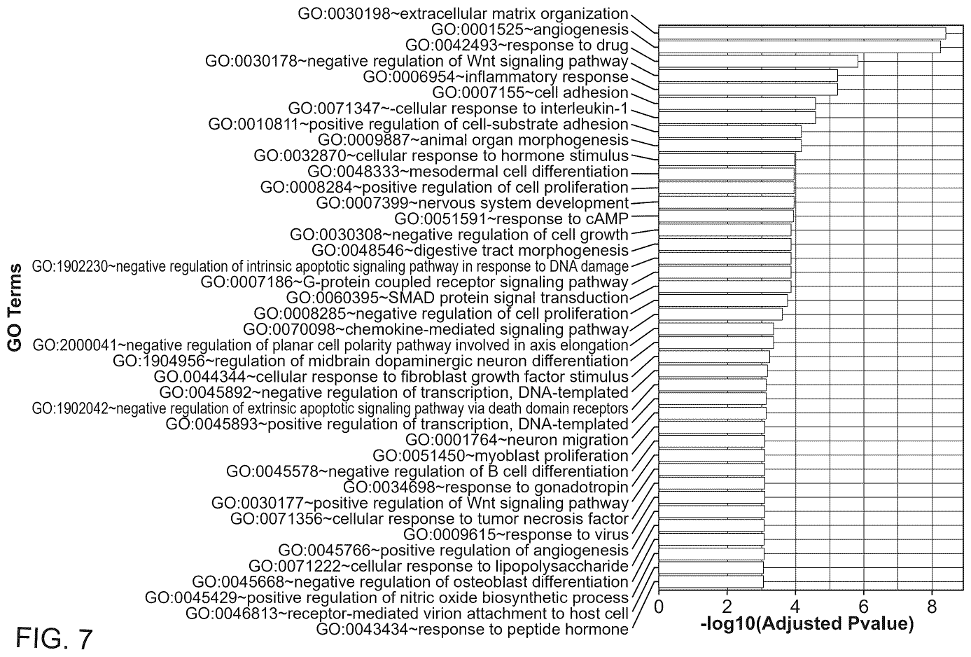

Comparison of the transcriptome of ASCs propagated in xenofree media with growth factor cocktail to reference ASCs propagated in xenofree media revealed various upregulated and downregulated genes. Differentially expressed genes affect pathways like extracellular matrix organization, angiogenesis, nerve system development, and response to drug, amongst others (FIG. 7).

### Conclusion

The transcriptome of ASCs propagated in xenofree media with growth factor was determined. ASCs obtained from the optimized culture conditions express genes involved in regenerative processes including angiogenesis and neuroregeneration.

### Example 6—Expression of Angiogenic and Neurogenic Genes

This Example describes the determination of the increased expression of selected genes with regenerative potential by qPCR.

### Methods

Genes coding for surface markers or secreted factors that showed at least 2-fold increased or 2-fold decreased expression in cultures of all 3 donors with XF media plus growth factor cocktail were measured by qPCR (irrespective of statistical significance).

Upon specified passage, cells were harvested from culture in XF media with or without growth factor cocktail as described under culture experiments. Cells were washed in PBS and after complete removal of PBS, cell pellets are frozen at −80° C. RNA was isolated. RNA concentration and RNA purity was determined. cDNA was generated and qPCR was performed. Gene expression was reported relative to housekeeping genes B2M and TBP as 2\hat{ }−ΔCT values. Primers for qPCR are listed in Table 1. Included were samples from 3 donors, passage 5 grown in XF or XF plus growth factor cocktail; growth of 1 donor was repeated yielding 8 samples in total.

TABLE 1
Primers for TaqMan qPCR.

| Target | Assay ID |
|---|---|
| APOD | HS00155794_m1 |
| BDNF | Hs02718934_s1 |
| B2M | Hs00187842_m1 |
| CALCRL | HS00907738_m1 |
| CXCL8 | HS00174103_m1 |
| CXCL12 | Hs03676656_m1 |
| DKK3 | Hs00247429_m1 |
| EREG | Hs00914313_m1 |
| FGFR2 | Hs01552918_m1 |
| FGF2 | Hs00266645_m1 |
| GDNF | Hs01931883_s1 |
| HGF | Hs00300159_m1 |
| ICAM1 | Hs00164932_m1 |
| ICAM5 | Hs00170285_m1 |
| IGFBP2 | Hs10040719_m1 |
| NRCAM | Hs01031598_m1 |
| PECAM1 | Hs01065279_m1 |
| SEMA3A | Hs00173810_m1 |
| TBP | Hs00427620_m1 |
| TMEFF2 | Hs01086902_m1 |
| VEGFA | Hs00900055_m1 |

### Results

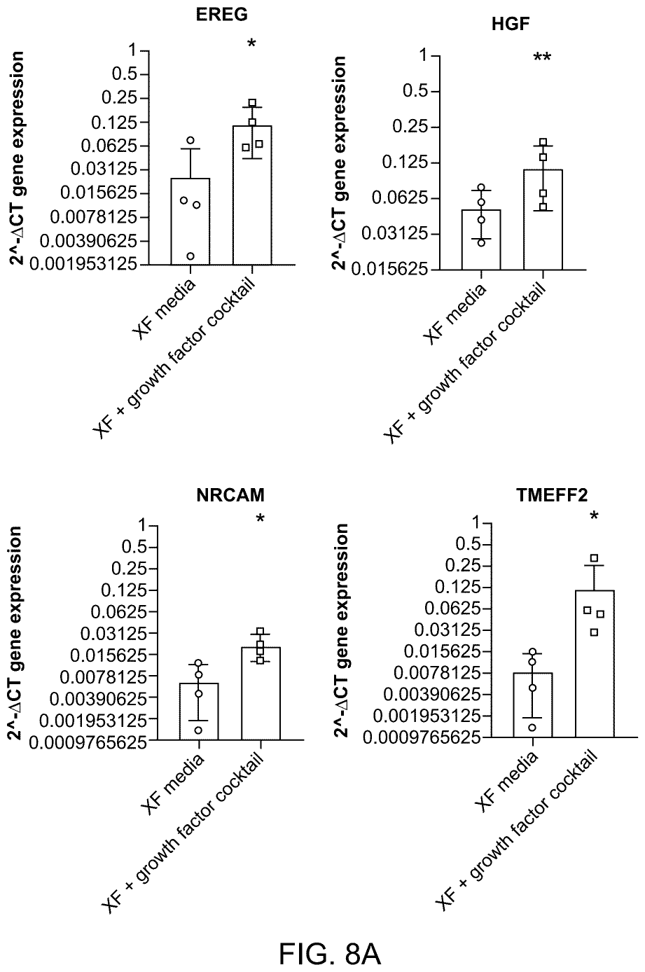

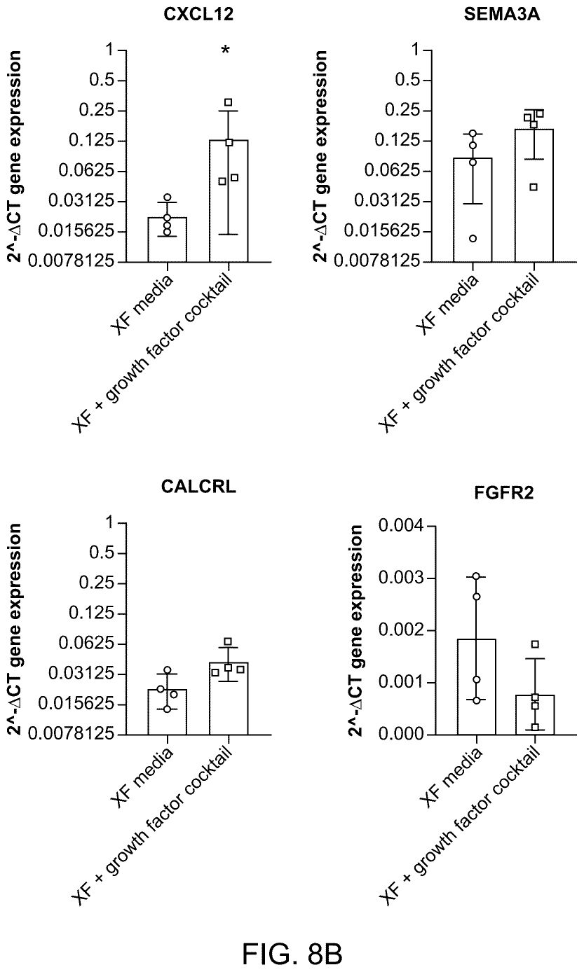

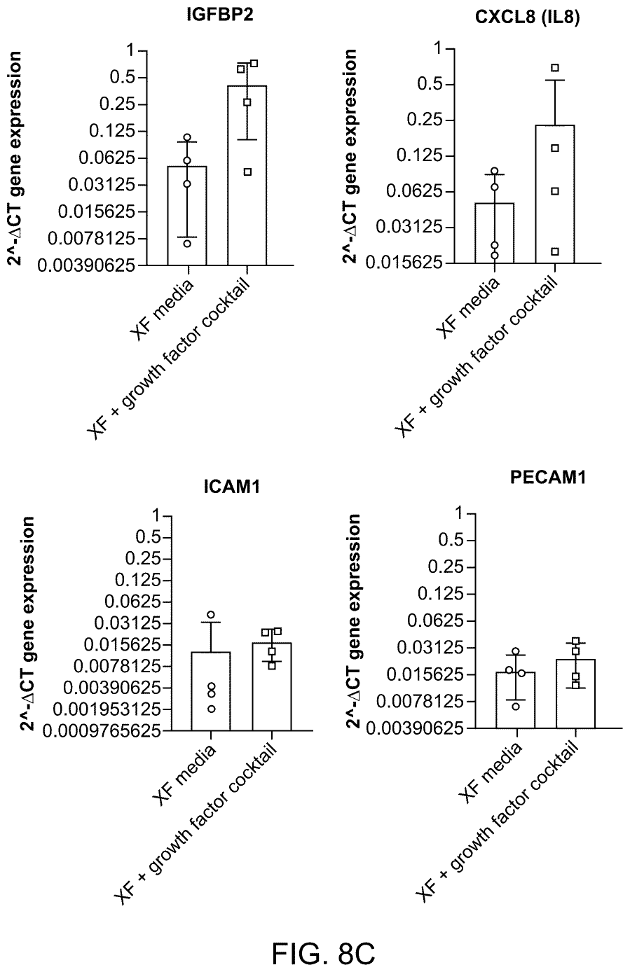

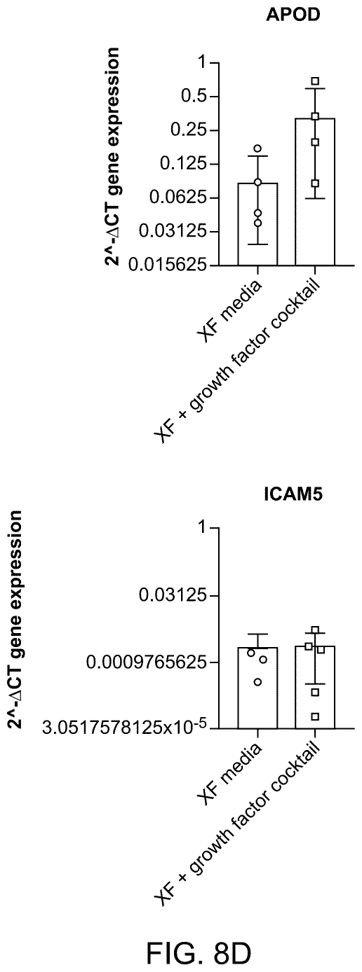

Having identified a list of relevant genes, another round of cells was cultured to passage 5 in either XF media or XF media containing the growth factor cocktail described in Example 1. The expression of selected genes through qPCR was determined. The expression of these proangiogenic and pro-neurogenic molecules was accompanied by the confirmed expression of surface molecules NRCAM and TMEFF2. Data is seen in FIGS. 8A-8D.

### Conclusion

The ASCs have an expression profile that features proangiogenic and neuroregenerative secreted factors.

### Example 7—Secreted Factors and Surface Marker Expression During Culture in Growth Factor Containing Media

This Example describes characteristics of cells cultured using the methods described herein.

### Methods

Passage 2 cells were thawed and cultured as described in the growth experiments to passage 8 or 16. RNA was isolation and qPCR was performed as described in Example 6 using primers as listed in Table 1. For statistical analysis, two-way ANOVA with Dunnett method to compare means of different passages to passage 3 was performed.

For detection of DKK3 in supernatant, cells grown to passage 3 or 8 as described in growth experiment were thawed and recovered in cultured medium. After 24 hours, culture medium was exchanged to starvation medium that did not contain any growth factors and supplements. Supernatant from starvation cultures was harvested after 5 hours and frozen. DKK3 concentration was determined after thawing of supernatant by ELISA. Absorbance measurements were performed.

### Results

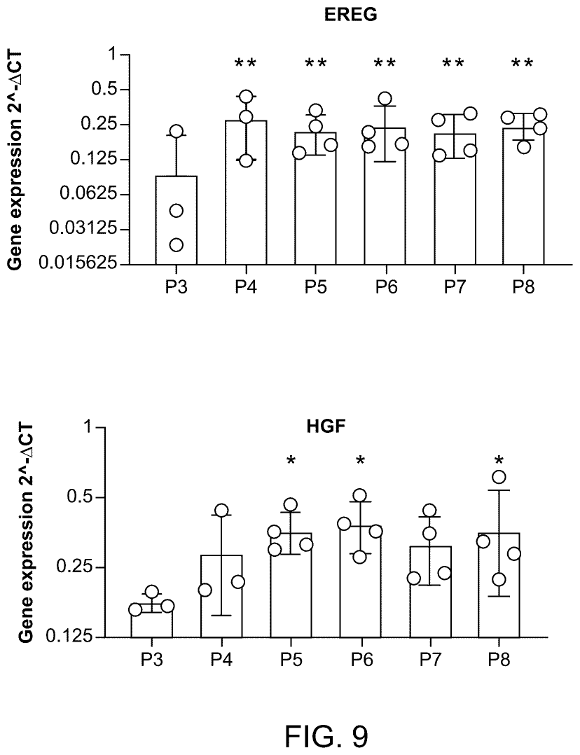

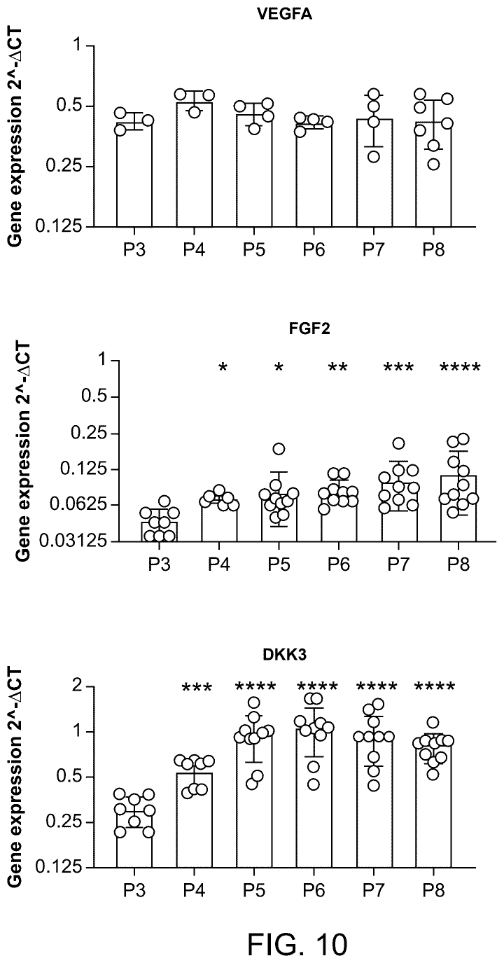

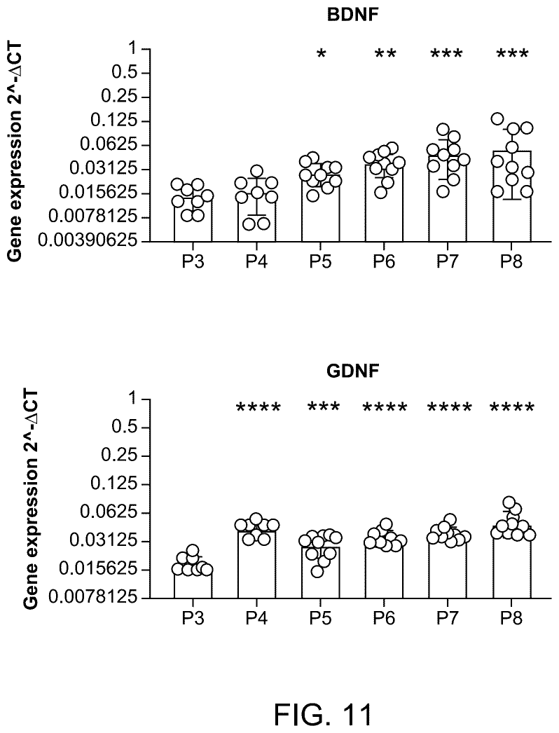

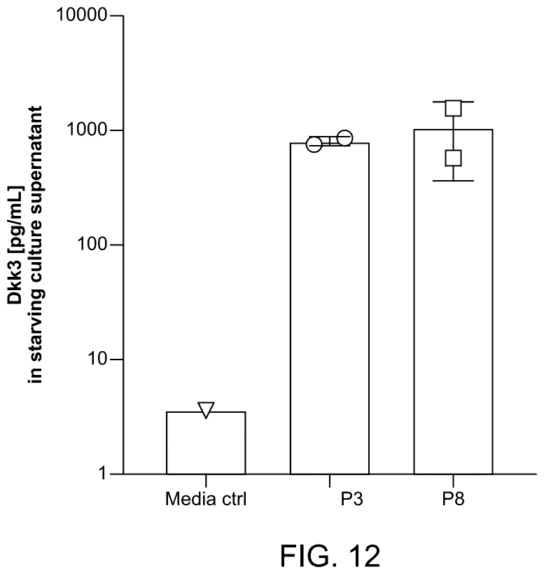

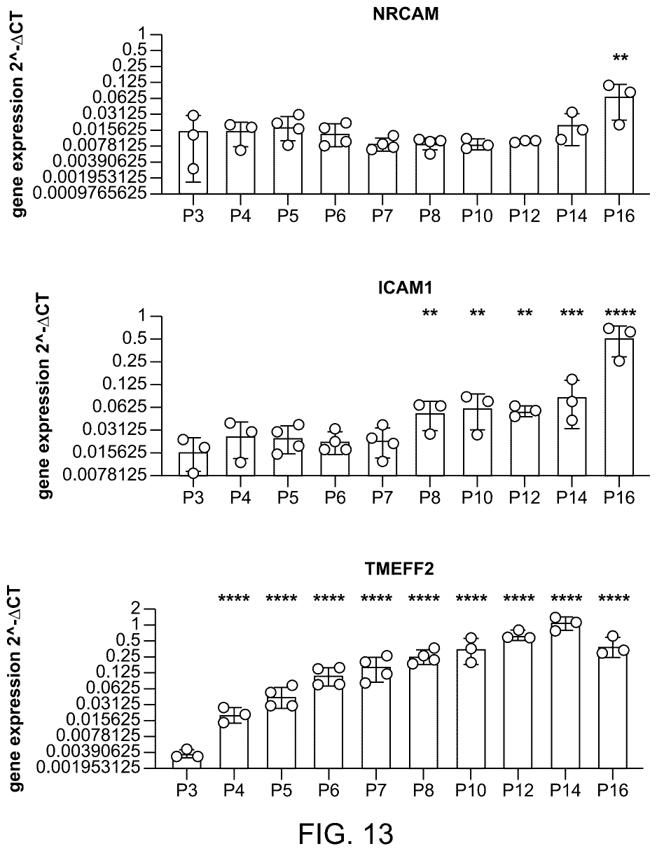

ASCs propagated in xenofree medium with the growth factor cocktail expressed angiogenic secretory factors EREG and HGF (FIG. 9). The expression of these factors was accompanied by other angiogenic factors like VEGFA, FGF2, and DKK3 (FIG. 10) as well as by neurotrophic secretory factors BDNF and GDNF (FIG. 11). The cells secreted high amounts of proangiogenic and neuroprotective DKK3 (FIG. 12). Surface molecules like NRCAM, ICAM1, or TMEFF2 increase with time in culture (FIG. 13).

### Conclusion

ASCs obtained from optimized culture conditions are characterized by ability to express and secrete angiogenic and neurotrophic factors.

### Example 8—Angiogenic Functionality of Cell Preparations from Tissue Culture

This Example describes determining the angiogenic functionality of cell preparations from tissue culture.

### Methods

### Matrigel Plug/In Vivo Angiogenesis

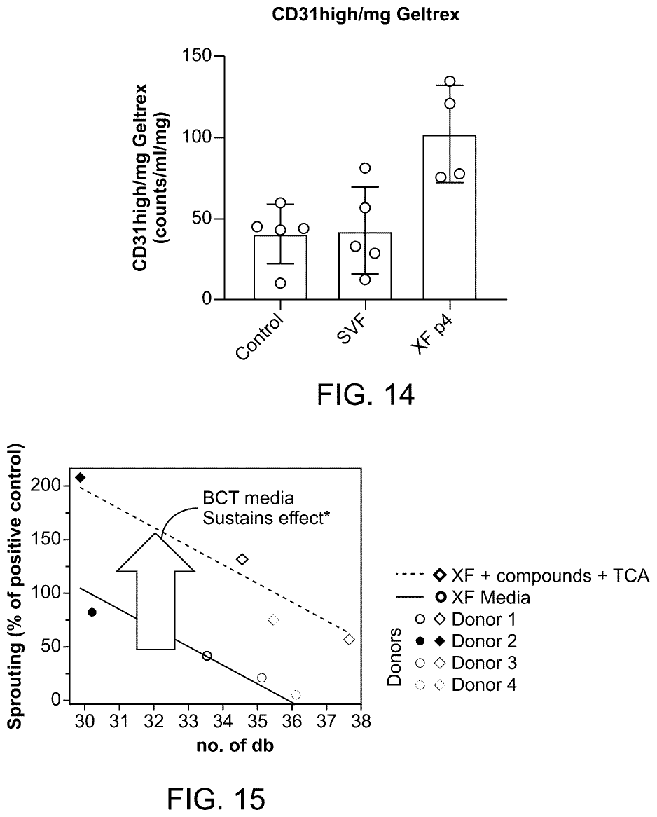

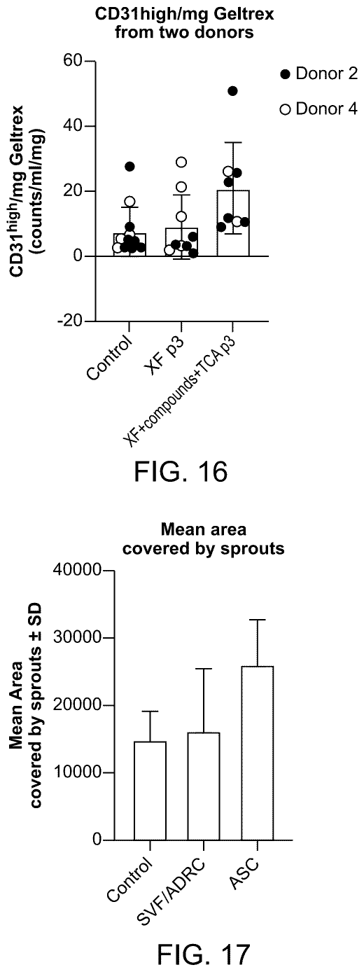

10-week-old C57BL6/J female mice were injected subcutaneously into the flank with 500 μl ice-cold Geltrex at a final concentration of 10 mg/mL. The solution and instruments need to be kept on ice at time of injection, as the growth-factor reduced LDEV-free Geltrex (a basement membrane matrix solution produced by murine Engelbreth-Holm-Swarm (EHS) tumors) solidifies at body temperature. Geltrex were supplemented with various grown ADRCs or ASCs at a concentration of 2.5E6 cells/mL or DMEM as a negative control. After 5 (FIG. 14) or 10 days (FIG. 16), the animals were euthanized and Geltrex plugs were removed carefully. The plugs were digested for 2 h at 37° C. and 500 rpm shaking in an enzymatic digestive mixture consisting of 3000 U/ml Collagenase and 1000 U/ml Hyaluronidase, and 2667 U/ml DNase dissolved in low glucose DMEM. Following digestion, cells were filtered using a 40 μm nylon mesh. Cells were washed in FACS buffer and stained with murine CD31-PE and murine CD45-VioGreen antibodies to be able to distinguish endothelial cells from the hematopoietic population. Samples were analyzed by flow cytometry.

### Aortic Ring Assay

This assay is used to investigate if factors secreted by ASCs in culture will stimulate angiogenesis from pieces of aortas. 7-9-weeks-old C57BL6/J male mice were euthanized, and aorta was carefully extracted. Each aorta was cleaned for excess connective tissue under a microscope to ensure complete clean material.

After aortas were extracted, they were flushed out in the lumen with cold Heparin and cold media without growth factors (starvation media). Cleaned aortas were kept overnight in starvation media at 37 degrees, 5% CO2. Next day, aortas were cut into rings of 0.5 mm length and embedded in Geltrex diluted with starvation media (7.5 mg/mL final protein concentration). The plates used for the assay was “integrated discrete multiple organ co-culture” (IdMOC) technology, with 6 wells sharing the same media in a unit called a chamber, the plates thus allow for testing of paracrine signaling. The plates used for these experiments had 12 chambers, thus, a total of 96 wells. 3 of 6 wells in each chamber was filled with Geltrex and aortic rings. Immediately after, the plate was transferred to an incubator for the Geltrex to solidate. After Geltrex solidification, the remaining 3 wells was filled with starvation media and incubated overnight at 37 degrees, 5% CO2. Next day, the 3 wells with media were emptied, and 3,500 ASCs per well were seeded. Therefore, each chamber will have 3 aortic rings (from 3 different mice) and either 3 wells of ASCs, or 3 empty wells, acting as controls. The individual ASC populations have been subjected to different treatments before initiation of the aortic ring assay. For instance, treatment with xenofree media, and compounds as described herein. Afterwards, ASCs were allowed to attach to the plate for 5 hours. Then, media was removed and the chambers filled with media, to bring individual wells into fluid contact for the first time. All chambers with cells were supplemented with 0.5% FBS starvation media. There were no cells in the negative- or positive control chambers, just aortic rings with either 0.5% or 2% FBS in starvation media, respectively.

Cells were cultured together with the rings for 5 days. Subsequently, sprouts from aortic rings were imaged by microscope with phase contrast to detect the complexity of the sprouts. Images were analyzed by Image J to quantify mean area covered by the sprouts.

### Corpus Cavernosum Explant Assay

Another version of the above-mentioned assay with Aortic rings was further developed using penile instead of aortic tissue. This assay is also used to investigate if factors secreted by ASCs in culture will stimulate angiogenesis from penile tissue, specifically corpus cavernosum. 7-9-weeks-old C57BL6/J male mice were euthanized, and corpus cavernosum was carefully extracted. Each corpus cavernosum was cleaned for excess connective tissue under a microscope to ensure complete clean material. Cleaned corpora cavernosa were kept overnight in starvation media at 37 degrees, 5% CO2. Next day, corpora cavernosa were cut into rings of 0.5 mm length and embedded in Geltrex diluted with starvation media (7.5 mg/mL final protein concentration). The culture labware used for the assay was “μ-slide 2 well co-culture” (ibidi GmbH), with 9 spots for tissue slices sharing the same media in a unit called a well, the slides thus allow for testing of paracrine signaling. In 3 to 7 of the 9 spots, 1250 to 5000 ASCs per spot were seeded in their specific propagation media and incubated overnight at 37 degrees, 5% CO2. Next day, cells were washed thoroughly in PBS and starvation media was added to the cells. 2 to 4 of 9 spots in each chamber were filled with Geltrex and corpus cavernosum slices. Therefore, each chamber contained 3 to 7 spots with ASCs and 2 to 4 spots with slices from corpora cavernosa. The ASC populations have been propagated in xenofree media with growth factor cocktail. Immediately after, the plate was transferred to an incubator for the Geltrex to solidate. After Geltrex solidification, both wells were filled with starvation media containing 0.5% FBS to bring cells and corpus cavernosum into contact for the first time. Cells were cultured together with the slices for 4 to 5 days. Subsequently, sprouts from corpora cavernosa were imaged by microscope with phase contrast to detect the complexity of the sprouts. Images were analyzed by Image J to quantify mean area covered by the sprouts

### Results

Functionality of cultured cells was compared to functionality of cells from non-cultured stromal vascular fraction (SVF) isolate. Cell preparations generated from tissue culture were more effective to stimulate in vivo growth of murine CD31^{high} cells (endothelial cells) within Geltrex plugs compared to cell preparations from SVF isolate or DMEM control preparation (FIG. 14).

Next, cells from optimized culture conditions were compared to cells from standard culture. Cell preparations generated in XF media plus compounds plus TCA were more effective in stimulating ex vivo outgrowth of endothelial sprouts from murine aortas compared to cell preparations generated in XF media (FIG. 15). Furthermore, cell preparations generated in XF media plus compounds plus TCA were more effective to stimulate in vivo growth of murine CD31^{high} cells within Geltrex plugs compared to cell preparations generated in XF media only (FIG. 16). As cell preparations from XF media cultured cells are more effective than SVF isolate (FIG. 14), it can be concluded that preparations from XF media plus compounds plus TCA are more effective than SVF isolate. Finally, cell preparations generated in XF media plus compounds plus TCA were more effective to stimulate ex vivo outgrowth of endothelial sprouts from murine corpus cavernosum compared to preparations from SVF isolate (FIG. 17).

### Conclusion

Cell preparations generated in XF media plus compounds plus TCA demonstrated angiogenic functionality.

### Example 9—BCT Cell Type is a New Donor-Independent Cell Type with No Natural Occurrence

This Example describes the production of BCT cells as a unique cell type distinct from donor origin.

### Methods

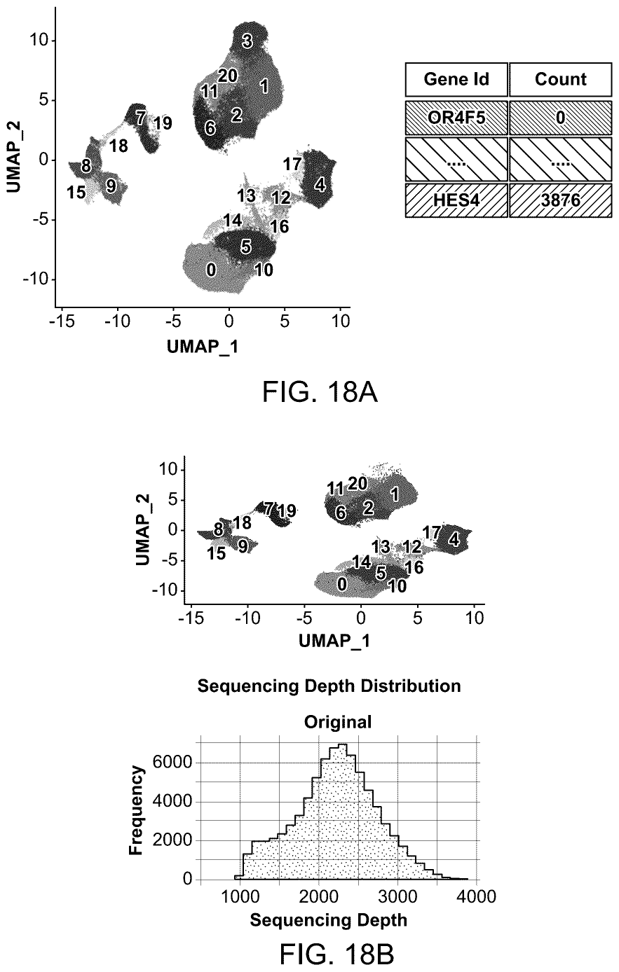

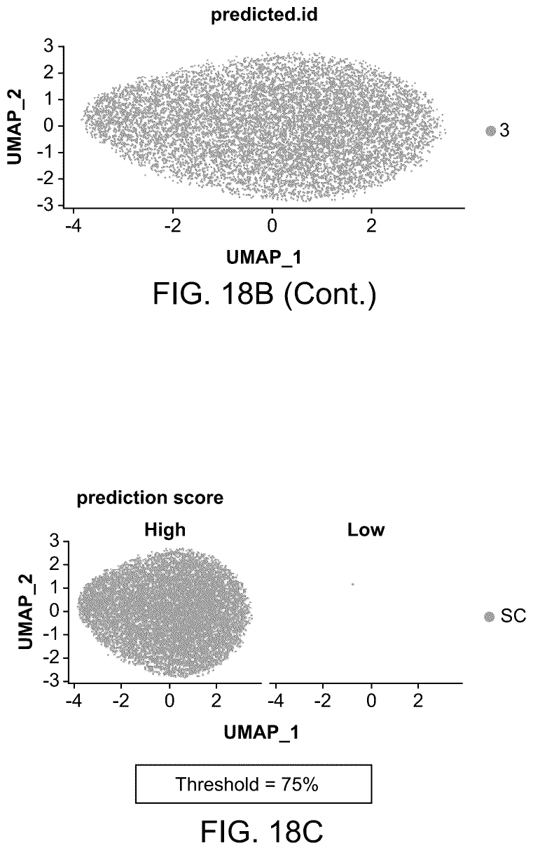

Step 1—Adipose Tissue Harvest and Cell Dissociation: Freshly harvested deep subcutaneous adipose tissue obtained by liposuction was maintained at 4° C. until further processing (up to 24 hours). Initial processing involved washing the adipose tissues five times with Ringer's lactate. Tissue was digested with collagenase in Ringer's lactate on rocker at 38° C. Enzymatic digestion was stopped by the addition of cell culture media containing 10% FBS. Cell solution was centrifuged, fat layer, oil and tumescent fluid were removed and washed in Ringer's lactate. Pellet was dissolved in media, cells were filtered through a 100 μm filter, subsequently through a 30 μm filter, and cells were counted. Buffer was exchanged to cryopreservation buffer and cells were frozen.
Step 2—10× Library Preparation, Sequencing, and Alignment: Selected samples (n=7) were provided for single-cell RNA-sequencing analysis. Single-cell RNA-sequencing libraries were prepared using the Chromium Single Cell 3′ assay. Libraries were sequenced with 2×150 read length. Raw reads were aligned to the human genome (hg38). Individual samples were aggregated to generate a merged digital expression matrix.
Step 3—Cell Clustering and Cell Type Annotation: The cells were clustered in a merged matrix. Cells with fewer than 300 or more than 6000 transcripts detected, or more than 15% mitochondrial gene expression were filtered out as low-quality cells. Gene counts for each cell were normalized, scaled, and subjected to natural-log transformation. Variable genes were selected, and data scaling was performed. Principal component analysis (PCA) was conducted on variable genes, and 30 principal components were used for cell clustering (resolution=0.5) and Uniform Manifold Approximation and Projection (UMAP) dimensional reduction. Cluster markers were identified, and cell types were manually annotated based on these markers.
Step 4—Bulk RNA sequencing of BCT cells: Library was prepared via polyA selection and sequenced.
Step 5—Create pseudo bulk-RNAseq datasets from selected clusters: A pseudo bulk RNAseq dataset was created by taking a well-defined cluster in the SVF single cell data set and summarizing each gene read count from all cells in the cluster, producing a simulated RNAseq count matrix. Step 5 is summarized in FIG. 18A.
Step 6—Create pseudo-single cell RNAseq data from pseudo-bulk RNAseq data: A simulated (pseudo) single cell dataset was generated by mimicking the read count distribution of the SVF single-cell (SC) dataset. Using this distribution, reads were sampled from the count matrix of the bulk RNA-seq dataset, populating a Seurat element of 10,000 cells. Step 6 is summarized in FIG. 18B.
Step 7—Mapping pseudo-single cell RNAseq data to the native single cell data set: Using Seurat reference-based mapping methods with standard settings, two different datasets were integrated and compared by finding correspondences (anchors) between them. Step 7 is summarized in FIG. 18C.
Step 8—Trimming and QC of RNAseq data: For each of the three bulk RNA-seq dataset produced, the adapters were removed, the reads were aligned to the human genome, and a multiqc analysis was performed. This procedure produced a count matrix for each of the donors.
Step 9—Create pseudo-single cell RNAseq data from bulk RNAseq data: A pseudo-single cell Seurat object was created using the method described in Step 7 for each of the three count matrices.
Step 10—Mapping of pseudo-single cell RNAseq data to original SVF Single-Cell RNAseq data: Each of the three pseudo-single cell data sets were mapped to the SVF single cell RNAseq data set originating from the previous steps with the same procedure as described in Step 7.
Step 11—Investigating similarity of pseudo-single cell donor data sets: Each of the three pseudo-single cell data sets were mapped to each other with the same procedure as described in Step 7.
Step 12—Mapping pseudo-single cell RNAseq data sets to a public dataset: Each of the three pseudo-single cell data sets were mapped to a public data set containing expression data of cell types in adipose tissue similar to the raw material used for producing BCT cells in Step 4.
Step 13—Cluster analysis of the pooled set of pseudo-single cell RNAseq data: The three sets of pseudo-single cell RNA-seq datasets were integrated using Seurat's anchor-based integration method, followed by clustering and UMAP visualization.
Step 14—Merge SVF single cell RNAseq data with pseudo-single cell RNAseq data and re-cluster the full data set: The pseudo-single cell RNA-seq datasets were merged with the SVF single cell dataset using the same integration method, followed by re-clustering and UMAP visualization.

### Results

FIGS. 18A-18C show the pseudo single cell RNA sequencing dataset generated from a pseudo bulk RNA sequencing dataset from a selected cluster mapped to this selected cluster when compared again to the full single cell RNA sequencing dataset. The high prediction score verifies that the concept of the pseudo single RNA sequencing dataset generated from bulk RNA sequencing data can be used to compare to SVF single-cell RNA sequencing data.

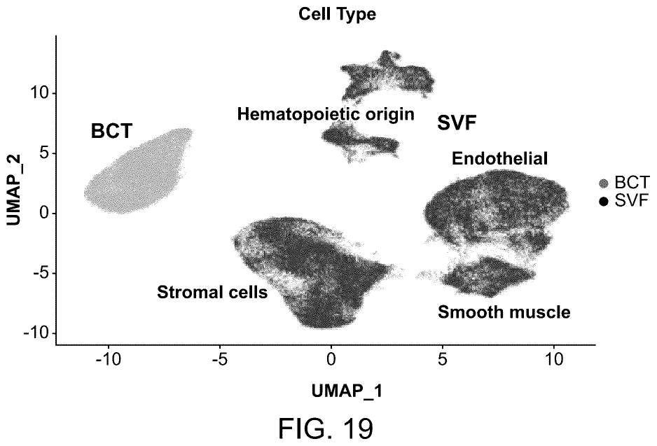

The three pseudo-single cell data sets did not map to any cluster in the original SVF Single-Cell RNAseq data set as seen in FIG. 19 and Table 2.

TABLE 2
Cell Mapping Data

| Donor | % cells with anchors | Could Map? | Mean mapping score |
|---|---|---|---|
| Donor 1 | 0.27 | FALSE | — |
| Donor 2 | 0.32 | FALSE | — |
| Donor 3 | 0.19 | FALSE | — |

All three pseudo-single cell data sets mapped to each other with high mapping scores, demonstrating that the produced BCT cells were all of the same cell type. Mapping the pseudo-single cell data sets to a public dataset concluded that the produced BCT cells could not be mapped to any cluster in the public data set. The integrated data from the pooled pseudo-single cell RNAseq datasets formed a distinct cluster separate from the SVF single cell dataset, indicating the produced BCT cells are significantly different from naturally occurring cell types from donor material.

### Conclusion

This Example demonstrate that the produced BCT cells are a unique cell type, independent of donor origin and without a natural occurrence in human adipose tissue.

### Example 10—Determination of Cell Characteristics of BCT Cells Compared to Alternative ASC Preparations from Identical Starting Material

This Example describes characterization of BCT cells prepared using xenofree media with growth factor cocktail/compounds as compared to alternative cell culture conditions.

### Methods

BCT Cell Culture: ADRCs were isolated from the lipoaspirate of 3 donors, as described in the above Examples. ASC preparation were generated by culturing the cells in xenofree media with growth factor cocktail/compounds (EGF (5 ng/mL), bFGF (10 ng/ml), IGF (20 ng/mL), VEGF (0.5 ng/ml), Ascorbic acid 1 μg/mL, hydrocortisone (0.2 μg/mL), TCA (10 μM)) up to passage 5 as described in the above Examples.

Alternative ASC preparations: Alternative culture conditions were used to generate ASC preparations with different profiles:
Alternative condition 1 (#2): ASC preparations were generated by culturing the cells in xenofree media with growth factor cocktail/compounds (EGF (5 ng/ml), bFGF (10 ng/ml), IGF (20 ng/ml), VEGF (0.5 ng/ml), Ascorbic acid 1 μg/mL, hydrocortisone (0.2 μg/mL), TCA (10 μM)) up to passage 2.
Alternative condition 2 (OG): ASC preparations were generated by culturing the cells in xenofree media with growth factor cocktail/compounds (EGF (5 ng/ml), bFGF (10 ng/ml), IGF (20 ng/ml), VEGF (0.5 ng/mL), Ascorbic acid 1 μg/mL, hydrocortisone (0.2 μg/mL), TCA (10 μM)) up to passage 5. For Alternative condition 2, cells were grown for an extended period of 7 days, allowing for increased cell numbers/cm² culture dish/increased cell densities/increased confluency beyond the normal cell density (i.e., greater than 80% confluency).
Alternative condition 3 (SS): ASC preparations were generated by culturing the cells in xenofree media with growth factor cocktail/compounds (EGF (5 ng/ml), bFGF (10 ng/ml), IGF (20 ng/mL), VEGF (0.5 ng/mL), Ascorbic acid 1 μg/mL, hydrocortisone (0.2 μg/mL), TCA (10 μM)) up to passage 15.
Alternative condition 4 (CH): ASC preparations were generated by culturing the cells in xenofree media with growth factor cocktail/compounds (EGF (5 ng/ml), bFGF (10 ng/mL), IGF (20 ng/ml), VEGF (0.5 ng/mL), Ascorbic acid 1 μg/mL, hydrocortisone (0.2 μg/mL), TCA (10 μM)) up to passage 4. Harvested cells were suspended in StemPro chondrogenesis differentiation medium with the supplement and used to form microdroplets. Cells were kept in culture for 4 weeks while the differentiation medium was refreshed every 3 days.

Protein Extraction and Quantitative Proteomics: Following culture, cells were harvested by trypsinization, followed by washing in PBS. Cell solutions were adjusted to 2% SDS-Tris, 100 mM, pH 8.5, and sonicated at 4° C. for 15 minutes, then samples were boiled for 10 minutes at 95° C. The samples were centrifuged, and protein concentrations were determined using the BCA assay. For each sample, equal amounts of protein (100 ng) were taken for reduction, alkylation, and enzymatic digestion steps. Samples were reduced for 15 minutes at 55° C., alkylated for 30 minutes at room temperature, and subjected to in-solution digestion overnight with 100 μL digestion buffer (100 mM TEAB) containing Trypsin and Lys-C in a 1:50 (enzyme:protein) ratio. Samples were dried in a SpeedVac concentrator at 45° C. and resuspended. Samples were analyzed by mass spectrometry (MS). MS data were collected over a 100-1700 m/z range using the ‘long-gradient’ diaPASEF method.

Data Analysis: Protein abundance values per protein group for each sample were determined. The samples were clustered using the Jaccard index between samples. Hierarchical clustering based on the Jaccard index was performed. The samples were also clustered using the cosine similarity between samples. Dendograms from clustering, missing values, and the number of replicates per protein, and average abundance levels across replicates were determined. Differential abundance analysis was performed for the following contrasts: i) BCT cells vs. #2; ii) BCT cells vs. OC; iii) BCT cells vs. SS; and iv) BCT cells vs. CH.

Proteins that were present in all replicates of one sample but absent in all replicates of another sample or vice versa were identified. If the protein was present in all replicates of the first sample in a contrast but absent from all replicates of the second sample, it was identified as present vs. absent for that contrast. In the opposite situation, where a protein was absent from all replicates of the first sample in a contrast but present in all replicates of the second sample, it was identified as absent vs. present for that contrast. The proteins identified in the above analyses were annotated with the average abundance level in the samples where they were present.

The resulting data analysis identified a panel of N=76 protein markers (designated as Marker-A, Marker-B, Marker-C, Marker-D, Marker-E, etc.) that were:
1) Detected at high abundance in BCT cells across all three donors and >[LOG₂]3-fold higher compared to at least one of the alternative culture conditions tested. Compared to the CH condition, >[LOG₂]6-fold higher.
2) Detected at low abundance in BCT cells across all three donors and >[LOG₂]₃-fold lower compared to at least one of the alternative culture conditions tested. Compared to the CH condition, >[LOG₂]6-fold lower.
3) Present in BCT cells but not in one of the alternative culture conditions tested.
4) Absent in BCT cells but not in one of the culture conditions #2, OC, SS.
5) Detected at high abundance in BCT cells across all three donors and consistently upregulated or present compared to all alternative conditions, at least [LOG₂]2-fold upregulated for one of the alternative conditions.
6) Detected at low abundance in BCT cells across all three donors and consistently downregulated or absent compared to all alternative conditions, at least [LOG₂]2-fold downregulated for one of the alternative conditions.

### Results

Comparison of the proteome within the target BCT cells vs. ASC preparations from alternative culture conditions resulted in the following 76 markers that describe the characteristics of the target BCT cells as seen in Table 3.

TABLE 3
Protein markers of BCT cells

| Proteins | Regulation | In contrast to BCT |
|---|---|---|
| AOC3 | Absent | #2 |
| C2 | Absent | #2 |
| CFD | Absent | OC |
| CRIP1 | Absent | OC |
| F9 | Absent | SS |
| IGLV3-19 | Absent | #2 |
| NTNG1 | Absent | SS |
| SERPINB7 | Absent | SS |
| SNCA | Absent | SS |
| BGN | Downregulated | CH |
| CKB | Downregulated | SS |
| COL12A1 | Downregulated | CH |
| COL14A1 | Downregulated | CH; OC; SS |
| DCN | Downregulated | CH; OC |
| FBLN1 | Downregulated | CH; OC |
| FCN3 | Downregulated | OC |
| FUCA1 | Downregulated | SS; OC |
| GPLD1 | Downregulated | OC; SS; CH |
| GPNMB | Downregulated | SS; CH; #2; OC |
| HSPB6 | Downregulated | SS; OC |
| HTRA1 | Downregulated | CH |
| ICAM1 | Downregulated | SS |
| KRT86 | Downregulated | OC |
| LTF | Downregulated | SS |
| LUM | Downregulated | CH; OC |
| MX1 | Downregulated | OC; SS |
| POSTN | Downregulated | CH |
| RAB27B | Downregulated | SS; OC; CH |
| SERPINB2 | Downregulated | SS; #2; OC; CH |
| SOD2 | Downregulated | SS; OC |
| TGM2 | Downregulated | SS; OC |
| TNC | Downregulated | CH |
| ALDH2 | Downregulated | #2; OC; SS; CH |
| DPP4 | Downregulated | #2; OC; SS; CH |
| SECTM1 | Downregulated | #2; OC; SS; CH |
| AKR1C1 | Downregulated | #2; OC; SS; CH |
| ADAM15 | Present | OC |
| ADIRF | Present | CH |
| AMD1 | Present-Upregulated | OC; SS; CH |
| CDCA8 | Present-Upregulated | OC; CH; SS |
| CDKN2C | Present | SS |
| CHST11 | Present-Upregulated | OC; SS |
| CXCL5 | Present | #2; OC; CH |
| DKK2 | Present | OC; SS |
| GPALPP1 | Present-Upregulated | CH; SS |
| IGFBP2 | Present-Upregulated | #2; SS; CH |
| LRP11 | Present | SS |
| MEST | Present | #2; OC |
| MMP1 | Present | OC |
| MT2A | Present | CH |
| NEO1 | Present | SS |
| NPTX1 | Present | #2; OC |
| PRXL2A | Present | SS |
| SMOC1 | Present | OC |
| SRGN | Present | #2 |
| ST6GAL2 | Present | #2; OC; SS; CH |
| TNFRSF6B | Present | #2; SS; CH |
| TPPP3 | Present | SS |
| TRIAP1 | Present | CH |
| ULBP2 | Present | SS |
| VEGFD | Present | SS |
| CDK1 | Upregulated | OC; SS |
| F5 | Upregulated | SS |
| GINS3 | Upregulated | OC; SS; CH |
| KPNA2 | Upregulated | OC; SS |
| MKI67 | Upregulated | OC; SS |
| NCAPD2 | Upregulated | OC; SS |
| PBK | Upregulated | OC; SS |
| POLA1 | Upregulated | OC; SS; CH |
| PRG4 | Upregulated | #2; CH |
| SCUBE3 | Upregulated | #2; OC; CH |
| SPDL1 | Upregulated | OC; SS; CH |
| TOP2A | Upregulated | OC; SS |
| TYMS | Upregulated | OC; SS |
| UHRF1 | Upregulated | OC; SS |
| EFHD2 | Upregulated | #2; OC; SS; CH |

Table 4 contains markers that are expressed on the cell surface or secreted that can be used for cell characterization:

TABLE 4
Cell surface and secreted protein markers

| Proteins | Regulation | In contrast to BCT |
|---|---|---|
| AOC3 | Absent | #2 |
| C2 | Absent | #2 |
| CFD | Absent | OC |
| F9 | Absent | SS |
| IGLV3-19 | Absent | #2 |
| NTNG1 | Absent | SS |
| SNCA | Absent | SS |
| BGN | Downregulated | CH |
| CKB | Downregulated | SS |
| COL12A1 | Downregulated | CH |
| COL14A1 | Downregulated | CH; OC; SS |
| DCN | Downregulated | CH; OC |
| FBLN1 | Downregulated | CH; OC |
| FCN3 | Downregulated | OC |
| GPLD1 | Downregulated | OC; SS; CH |
| HSPB6 | Downregulated | SS; OC |
| HTRA1 | Downregulated | CH |
| LTF | Downregulated | SS |
| LUM | Downregulated | CH; OC |
| POSTN | Downregulated | CH |
| SERPINB2 | Downregulated | SS; #2; OC; CH |
| TGM2 | Downregulated | SS; OC |
| TNC | Downregulated | CH |
| CXCL5 | Present | #2; OC; CH |
| DKK2 | Present | OC; SS |
| IGFBP2 | Present-Upregulated | #2; SS; CH |
| MMP1 | Present | OC |
| NEO1 | Present | SS |
| NPTX1 | Present | #2; OC |
| SMOC1 | Present | OC |
| SRGN | Present | #2 |
| TNFRSF6B | Present | #2; SS; CH |
| ULBP2 | Present | SS |
| VEGFD | Present | SS |
| F5 | Upregulated | SS |
| SCUBE3 | Upregulated | #2; OC; CH |

### Example 11—ASCs for Treating Erectile Dysfunction

This Example describes the use of ASCs generated using the culture medium described herein for treating erectile dysfunction.

### Methods

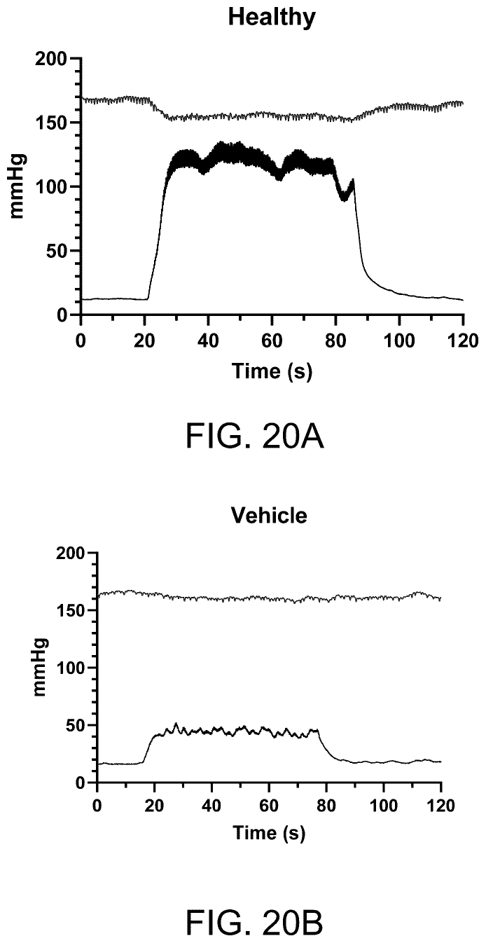

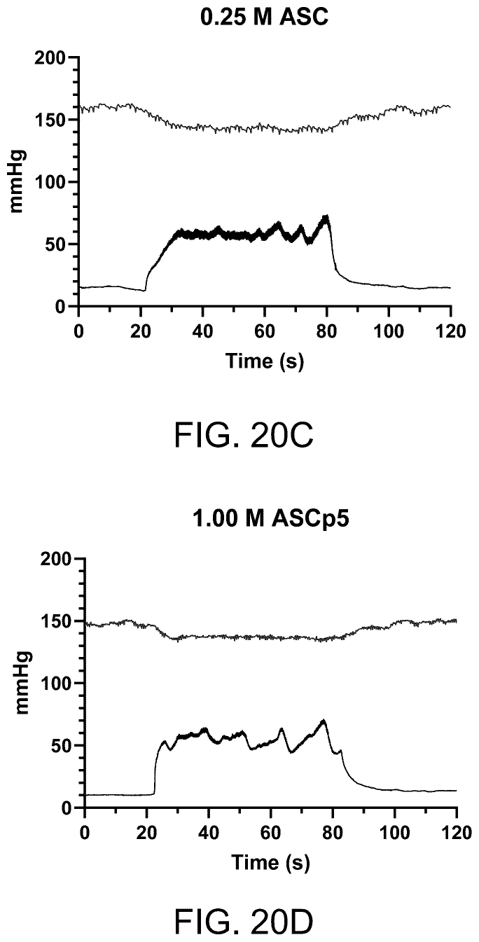

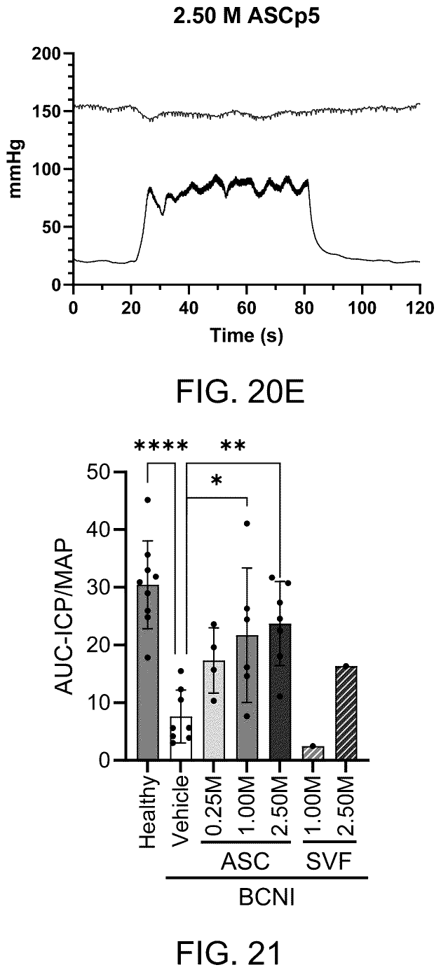

Erectile dysfunction rat model: Male Sprague Dawley rats (10 weeks old) were randomly allocated to either experimental or control groups. Under anesthesia, a midline laparotomy was performed to expose the pelvic cavity, and the major pelvic ganglion (MPG) was identified on the dorsal side of the prostate. The cavernous nerve (CN), just caudal to the MPG, was isolated by blunt dissection and subjected to a standardized crush injury using a designated microneedle holder, applying pressure 2×2 minutes. The procedure was repeated on the CN on the other side, thereby performing bilateral cavernous nerve injury (BCNI). Following closure of the abdominal incision, 200 μl of either cultured ASC, stromal vascular fraction (SVF) or vehicle was administered by intracavernous injection into the penile tissue using a 30G needle. The needle was withdrawn 1 minute post-injection, and gentle pressure applied to the injection site to prevent leakage. 14 days after surgery, the animals were re-anesthetized (ketamine/xylazine/midazolam cocktail, 75/10/5 mg/kg, respectively). Scientists performing the measurements were blinded towards the treatment. The carotid artery was surgically exposed and catheterized using a fluid-filled PE-50 tubing containing saline and heparin (100 U/ml). The pressure transduction system was calibrated to 30 cm H₂O in atmospheric pressure and converted to mmHg. Simultaneously, the prostate was re-exposed, and the MPG and CN were identified. Proximal to the site of nerve injury, the CN was re-exposed and a bipolar silver Teflon-coated electrode was hooked around the nerve. A separate incision was made in the scrotum to dissect the ischiocavernosus muscle and expose the tunica albuginea of the penile crus. Another fluid-filled PE-50 tubing containing saline and heparin (100 U/ml) was inserted through the tunica albuginea, ensuring parallel alignment with the cavernous body. Intracavernous and arterial pressure were recorded continuously. Cavernous nerve stimulations were performed using the following parameters: current at 1.5 mA, frequency at 16 Hz, voltage at 6 V, and pulse width at 5 millisecond, for 1 minute with a minimum rest period of 3 minutes. The first 2 stimulations were disregarded due to diminished responses. Examples of plots can be seen in FIGS. 20A-20E. For each animal, the average area under the curve (AUC) of the intracavernous pressure (ICP) from 3 stimulations was calculated and normalized to the corresponding mean arterial pressure (MAP), where MAP was determined as

$$MAP = \frac{SBP + 2 ( DBP )}{3} ,$$

where SBP representing systolic and DBP diastolic blood pressure. Each animal's result was represented as a single data point in FIG. 21.

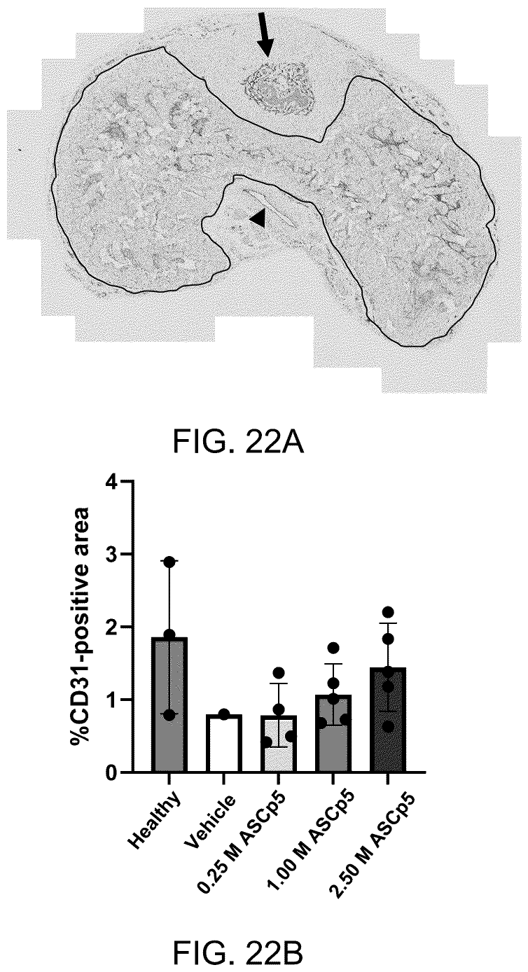

Semi-automated quantification of angiogenesis in penile tissue: Following completion of ICP and MAP measurements, penile tissues were excised and immediately fixed in 4% neutral buffered formalin for 3 days at 4° C. The fixed samples were then processed for paraffin embedding and oriented to obtain transverse sections of the penis. Immunohistochemical detection of endothelial cells was performed using a monoclonal anti-CD31 antibody applied at a 1:2000 dilution for 60 minutes. Signal detection was carried out for 32 minutes using the OmiMap-Rb-HRP detection reagent with DAB as the chromogen to produce a brown staining pattern, specifically targeting endothelial cells. The stained sections were digitally scanned and analyzed in a blinded manner. The corpus cavernosum region (indicated in black in FIG. 22A) was manually outlined, and three independent areas of background stainings were selected per slide. A custom image analysis script was used to define positively stained pixels as those exhibiting a higher brown intensity than the average intensity of the background regions. The proportion of positive pixels was then calculated as the percentage of total pixels within the corpus cavernosum area (FIG. 22B).

### Results

Treatment with ASCs at 1.0×10E6 and 2.5×10E6 cells significantly improved intracavernous pressure in the nerve injury-induced erectile dysfunction rats (BCNI) compared to vehicle controls (FIGS. 22A-22B). This demonstrates a dose-dependent regenerative effect of ASCs treatment, with the 2.5×10E6 cell dose providing the most pronounced improvement. Furthermore, ASC-treated animals exhibited a higher erectile response at a lower cell dosage compared to SVF-treated animals (FIGS. 22A-22B). On a molecular level, there is a dose-dependent increase in endothelial cells (CD31-positive cells) of ASC-treated animals compared to the vehicle-treated, an indication of new vessel growth induced by the ASCs (FIGS. 22A-22B).

### Conclusion

This Example shows that treatment with ASCs described herein restores erectile function after nerve injury in rats, supporting its angiogenic and neuroregenerative potential for nerve injury-induced erectile dysfunction.

What is claimed is:

1. A method of generating a plurality of adipose-derived stromal cells (ASCs) from adipose tissue, comprising:
(a) providing a cell preparation derived from the adipose tissue (an adipose-derived regenerative cell (ADRC) preparation) and a culture medium comprising from about 0.5 ng/mL to about 50 ng/mL VEGF, from about 0.5 ng/ml to about 50 ng/ml EGF, from about 1 ng/mL to about 100 ng/mL bFGF, from about 2 ng/ml to about 200 ng/ml IGF, from about 0.1 μg/mL to about 10 μg/mL Ascorbic acid, and from about 0.02 μg/mL to about 2 μg/mL hydrocortisone; and
(b) contacting the ADRC preparation with the culture medium for a time sufficient to grow mesenchymal stem cells (MSCs) in the ADRC preparation, thereby generating the ASCs.

2. The method of claim 1, further comprising enriching the MSCs and/or the ASCs from the ADRC preparation.

3. The method of claim 1, further comprising enriching factors or extracellular vesicles released from the ASCs, thereby providing a preparation of factors or extracellular vesicles released from the ASCs.

4. The method of claim 1, wherein the culture medium comprises: about 0.5 ng/ml VEGF, about 5 ng/mL EGF, about 10 ng/ml bFGF, about 20 ng/ml IGF, about 1 μg/mL Ascorbic acid, and about 0.2 μg/mL hydrocortisone.

5. The method of claim 1, further comprising one or more bile acids.

6. The method of claim 5, wherein the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA).

7. The method of claim 5, wherein the bile acid is taurocholic acid (TCA).

8. The method of claim 1, wherein the MSCs have increased expression and/or activity of angiogenic factors, neurogenic factors, or both angiogenic factors and neurogenic factors as compared to uncultured cells or cells cultured in a culture medium that does not comprise VEGF, EGF, bFGF, IGF, Ascorbic acid, and hydrocortisone or any functional fragments, variants, or homologous proteins of VEGF, EGF, bFGF, or IGF.

9. An adipose-derived stromal cell (ASC) preparation, produced by a process comprising:
(a) providing a cell preparation derived from adipose tissue (an adipose-derived regenerative cell (ADRC) preparation) and a culture medium comprising from about 0.5 ng/ml to about 50 ng/mL VEGF, from about 0.5 ng/ml to about 50 ng/mL EGF, from about 1 ng/ml to about 100 ng/mL bFGF, from about 2 ng/mL to about 200 ng/ml IGF, from about 0.1 μg/mL to about 10 μg/mL ascorbic acid, and from about 0.02 μg/mL to about 2 μg/mL hydrocortisone; and
(b) contacting the ADRC preparation with the culture medium for a time sufficient to grow mesenchymal stem cells (MSCs) in the ADRC preparation, thereby producing the adipose-derived stromal cells (ASCs).

10. The ASC preparation of claim 9, further comprising enriching the MSCs and/or the ASCs from the ADRC preparation.

11. The ASC preparation of claim 9, further comprising enriching factors or extracellular vesicles released from the ASCs, thereby providing a preparation of factors or extracellular vesicles released from the ASCs.

12. The ASC preparation of claim 9, wherein the culture medium comprises: about 0.5 ng/mL VEGF, about 5 ng/ml EGF, about 10 ng/mL bFGF, about 20 ng/ml IGF, about 1 μg/mL Ascorbic acid, and about 0.2 μg/mL hydrocortisone.

13. The ASC preparation of claim 9, further comprising one or more bile acids.

14. The ASC preparation of claim 13, wherein the bile acid is selected from the group consisting of Sodium glycocholate hydrate (GCA), Sodium glycochenodeoxycholate (GCDCA), Sodium taurochenodeoxycholate (TCDCA), Sodium glycodeoxycholate (GDCA), Sodium taurodeoxycholate hydrate (TDCA), Tauro-α-muricholic Acid Sodium Salt (TaMCA), and Sodium tauroursodeoxycholate (TUDCA).

15. The ASC preparation of claim 13, wherein the bile acid is taurocholic acid (TCA). The method of claim 1, wherein the MSCs have increased expression and/or activity of angiogenic factors, neurogenic factors, or both angiogenic factors and neurogenic factors as compared to uncultured cells or cells cultured in a culture medium that does not comprise VEGF, EGF, bFGF, IGF, Ascorbic acid, and hydrocortisone or any functional fragments, variants, or homologous proteins of VEGF, EGF, bFGF, or IGF.
